(19) United States
(12) Patent Application Publication
(10) Pub. No.: US 2023/0091561 A1
(43) Pub. Date: Mar. 23, 2023

(54) METHODS OF TREATING PRIMARY PROGRESSIVE MULTIPLE SCLEROSIS USING AN INHIBITOR OF BRUTON'S TYROSINE KINASE

(71) Applicants: Genentech, Inc., South San Francisco, CA (US); Hoffmann-La Roche Inc., Little Falls, NJ (US)

(72) Inventors: Hideki GARREN, Palo Alto, CA (US); Edmond Huatung TENG, San Mateo, CA (US); Aurelien VIACCOZ, Basel (CH); Hans-Christian VON BUEDINGEN, Basel (CH)

(73) Assignees: Genentech, Inc., South San Francisco, CA (US); Hoffmann-La Roche Inc., Little Falls, NJ (US)

(21) Appl. No.: 17/905,121

(22) PCT Filed: Feb. 25, 2021
(86) PCT No.: PCT/US2021/019502

§ 371 Date: Aug. 26, 2022

Related U.S. Application Data
(60) Provisional application No. 63/051,756, filed on Jul. 14, 2020.
(60) Provisional application No. 62/982,872, filed on Feb. 28, 2020.

(51) Int. Cl. A61K 31/519; C07K 16/28; A61K 9/20; A61K 9/48

(52) U.S. Cl. CPC A61K 31/519; C07K 16/2887; A61K 9/20; A61K 9/48

(57) ABSTRACT

Provided herein are methods of treating Primary Progressive Multiple Sclerosis (PPMS) in a subject in need thereof, by administering to the subject about 200 mg of fenebrutinib twice daily, or an equivalent amount of a pharmaceutically acceptable salt thereof.

### CROSS-REFERENCE TO RELATED APPLICATIONS

This application claims the benefit of priority of U.S. Provisional Application Ser. No. 63/051,756 filed Jul. 14, 2020, and U.S. Provisional Application Ser. No. 62/982,872 filed Feb. 28, 2020, the disclosures of both of which are incorporated herein by reference in their entireties.

### FIELD

The present disclosure relates to methods of treating primary progressive multiple sclerosis (PPMS) using an inhibitor of Bruton's tyrosine kinase (BTK).

### BACKGROUND

Bruton's Tyrosine Kinase (BTK): Discovery of the genetic basis for primary immunodeficiencies has been the source of new therapeutic targets in immunomodulatory therapies. In humans, mutations in the gene for Bruton's tyrosine kinase (BTK), which is located on the X chromosome, can result in the development of an immunodeficiency state characterized by a significant absence of circulating B cells (Bruton O C. Pediatrics 1952, 9:722-8; Conley M E. et al, Immunol Rev 2005, 203:216-34), and very low immunoglobulin levels due to a defect in B-cell differentiation at the pro- to pre-B cell stage that precludes assembly of the B-cell receptor (BCR) complex and immunoglobulin gene expression (Reth M, Nielsen P., Adv Immunol 2014, 122:129-75. doi: 10.1016/B978-0-12-800267-4.00004-3). Affected male patients have a primary immune deficiency, X-linked agammaglobulinemia (XLA), and are susceptible to recurrent infections starting shortly after birth. Patients with XLA can live relatively normal lives on a standard therapy of intravenous (IV) immunoglobulin, which suggests that BTK can be safely inhibited, especially in people with established immune systems. IV immunoglobulin replacement therapy lowers the rate of infection, reduces hospitalization rates for patients with XLA, and has greatly improved the long-term prognosis of these patients.

BTK is essential for the differentiation and activity of B cells during immune system ontogeny and normal adaptive immune responses. BTK is activated by phosphatidylinositol 3-kinase-dependent plasma membrane recruitment and phosphorylation on tyrosine Y551 by the Src-family kinase Lyn. Autophosphorylation and activation also occurs on tyrosine Y223 in a BTK-specific manner. Once activated, BTK induces PLCγ2- and Ca²⁺-dependent signaling, which leads to the activation of NF-κB- and NFAT-dependent pathways, this in turn leads to cellular activation and differentiation (Niiro H, Clark E A., Nat Rev Immunol 2002, 2:945-56). In addition, BTK is important in FcεRI signaling in both basophils and mast cells. BTK null mice have impaired FcεRI signaling resulting in decreased histamine and inflammatory cytokine release (Iyer A S, et al., J Bio Chem 2011, 286:9503-13. doi: 10.1074/jbc.M110.1656131).

Multiple Sclerosis: Multiple sclerosis (MS) is a chronic, inflammatory, demyelinating, and degenerative disease of the CNS that affects approximately 900,000 people in the United States (Wallin et al. 2019) and 2.3 million worldwide (GBD 2016 Multiple Sclerosis Collaborators 2019). It is primarily a disease of young adults, with 70%-80% of patients having an age of onset (i.e., initial clinical presentation to a physician) between 20 and 40 years (Anderson et al. 1992; Noonan et al. 2002) and has a gender bias influenced by the phenotype, with approximately up to 64%-70% of diagnosed patients being women (Anderson et al 1992; Noonan et al. 2002).

MS is classified into three clinical phenotypes, one of which is primary progressive (PPMS). PPMS is further subdivided into active and non-active forms based on the presence or absence of disease activity, defined by the presence of clinical relapses and/or gadolinium-enhancing lesions on T1-weighted magnetic resonance imaging (MRI) scan (T1Gd+) or new/enlarging T2-weighted lesions on MRI scan.

Although the mechanisms associated with disease progression are assumed to be present from the onset of the disease (Cree et al. 2019), clinical disability progression manifests often later in the course of a patient's disease most likely due to the degree of brain reserve of the patient. The symptomatic worsening associated with MS disability progression results in a slow, insidious loss of a patient's motor and sensory function, as well as cognitive decline and autonomic dysfunctions (Lassmann, 2018).

Without wishing to be bound by any theory, disability progression across the spectrum of MS might occur as a result of two concurrent inflammatory mechanisms, active inflammation and chronic compartmentalized inflammation. Chronic compartmentalized inflammation, which is driven by microglia activation, is associated with ongoing disability accumulation. Chronic compartmentalized inflammation is responsible for an increase in disability that occurs independently from relapses or disease activity and is characterized by demyelination and axonal loss (progression biology; Lassmann et al. 2019). Progressive forms of MS (PMS), including PPMS, are associated with a chronic and slow accumulation of T cells and B cells without leakage of the blood brain barrier and is believed to create subpial-demyelinated lesions in the cerebral and cerebellar cortex, as well as a slow expansion of pre-existing lesions in the white matter and diffuse chronic inflammation in the normal appearing white and gray matter (Lassmann 2018).

In vitro cell-based experiments suggest that antagonism of BTK with fenebrutinib leads to inhibition of BCR-dependent B-cell proliferation and a reduction of inflammatory cytokine production from myeloid cells (including tumor necrosis factor-α [TNF-α]). Myeloid effector functions are triggered by immune complexes in vitro and increasing evidence suggests that B cells and myeloid/microglia may be central to the immunopathology of MS. BTK inhibition has direct effects on myeloid lineage cells. As a result, there is a potential for BTK inhibition to affect microglia that are associated with the pathological hallmark of MS disease progression independent of relapse.

Even though there are many drugs currently available that target the pathological inflammatory mechanisms associated with relapses and relapse-associated worsening, currently only one is indicated for PPMS. As a result, the salient feature of disability progression in all forms of MS remains under addressed, and treatments that can stop or delay MS disease progression represent a serious unmet medical need.

### SUMMARY OF THE DISCLOSURE

Provided herein are methods and uses of a BTK inhibitor, fenebrutinib, or a pharmaceutically acceptable salt of fenebrutinib, for treating Primary Progressive Multiple Sclerosis (PPMS).

E1. In a first embodiment (Embodiment 1, “E1”), provided herein is a method of treating primary progressive multiple sclerosis (PPMS) in a subject in need thereof, comprising administering to the subject about 200 mg fenebrutinib twice daily, or an equivalent amount of a pharmaceutically acceptable salt thereof.

E2. The method of E1, further comprising evaluating disability progression in the subject.

E3. The method of E2, wherein disability progression is evaluated using the Expanded Disability Status Scale (EDSS), the 9-Hole Peg Test (9-HPT), or the Timed 25-Foot Walk Test (T25FWT), or any combinations thereof.

E4. The method of E2 or E3, comprising evaluating the onset of composite 12-week confirmed disability progression (cCDP12), wherein onset of the cCDP12 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT,
and wherein the progression event is confirmed at least 12 weeks after the initial progression.

E4a. A method of reducing the risk of experiencing cCDP12 in a subject with PPMS, comprising administering to the subject about 200 mg fenebrutinib twice daily, or an equivalent amount of a pharmaceutically acceptable salt thereof.

E4b. The method of E4a, wherein cCDP12 comprises the first occurrence of a progression event in the subject after beginning of administration of fenebrutinib or a pharmaceutically acceptable salt thereof, wherein the progression event is confirmed at least 12 weeks after the initial disability progression.

E4c. A method of reducing time to onset of cCDP12 in a subject with PPMS, comprising administering to the subject about 200 mg fenebrutinib twice daily, or an equivalent amount of a pharmaceutically acceptable salt thereof, wherein time to onset of cCDP12 comprises the period from before beginning administration of fenebrutinib or a pharmaceutically acceptable salt thereof to the first occurrence of a progression event, wherein the progression event is confirmed at least 12 weeks after the initial disability progression.

E4d. The method of E4b or E4c, wherein the progression event is one of:
(a) an increase from baseline in Expanded Disability Status Scale (EDSS) score of ≥1.0 point in a subject with a baseline EDSS score of 55.5 or an increase of ≥0.5 points in a subject with a baseline EDSS score of >5.5 (confirmed disability progression [CDP]);
(b) ≤20% increase from baseline in the Timed 25-Foot Walk Test (T25FWT); or
(c) ≤20% increase from baseline in time to complete the 9-Hole Peg Test (9-HPT).

E5. The method of any one of E2 to E4d, comprising evaluating the onset of 12-week confirmed disability progression (CDP12) in the subject, wherein the onset of CDP12 comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points; and wherein the progression of EDSS is confirmed at least 12 weeks after the initial progression.

E6. The method of any one of E2 to E5, comprising evaluating the onset of composite 24-week confirmed disability progression (cCDP24), wherein the onset of cCDP24 comprises at least one progression event selected from the group consisting of:
(a) an increase from baseline in EDSS score of at least 1.0 point in a subject with a baseline EDSS score of less than or equal to 5.5 points, or an increase from baseline in EDSS score of at least 0.5 point in a subject with a baseline EDSS score of greater than 5.5 points;
(b) increase from baseline of at least 20/a in time to complete the 9-HPT; and
(c) increase from baseline of at least 20% in T25FWT.
and wherein the progression event is confirmed at least 24 weeks after the initial progression.

E7. The method of any one of E2 to E6, comprising evaluating the onset of 24-week confirmed disability progression (CDP24) in the subject, wherein the onset of CDP24 comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points; and wherein the progression of EDSS is confirmed at least 24 weeks after the initial progression.

E8. The method of any one of E1 to E7, wherein time to a progression event in the subject is increased, wherein the progression event is:
an increase from baseline in EDSS score of at least 1.0 point in a subject with a baseline EDSS score of less than or equal to 5.5 points; or
an increase from baseline in EDSS score of at least 0.5 point in a subject with a baseline EDSS score of greater than 5.5 points.

E9. The method of any one of E1 to E8, wherein time to a progression event in the subject is increased, wherein the progression event is an increase from baseline of at least 20% in time to complete the 9-HPT.

E10. The method of any one of E1 to E9, wherein time to a progression event in the subject is increased, wherein the progression event is an increase from baseline of at least 20% in T25FWT.

E11. The method of any one of E1 to E10, wherein time to onset of CDP12 is increased.

E12. The method of any one of E1 to E11, wherein time to onset of cCDP12 is increased.

E13. The method of any one of E1 to E12, wherein time to onset of CDP24 is increased.

E14. The method of any one of E1 to E13, wherein time to onset of cCDP24 is increased.

E15. The method of any one of E8 to E14, wherein the increase is in comparison to a subject with PPMS that is not administered fenebrutinib or a pharmaceutically acceptable salt thereof.

E16. The method of any one of E8 to E15, wherein the increase is in comparison to a subject with PPMS who is administered an anti-CD20 antibody.

E17. The method of any one of E8 to E16, wherein the increase is at least 5%, at least 10%, at least 15%, at least 20%, at least 25%, at least 30%, or at least 35%.

E18. The method of any one of E1 to E17, wherein the progression of PPMS in the subject is slowed.

E19. The method of any one of E1 to E18, wherein the onset of at least one progression event in the subject is delayed.

E20. The method of any one of E1 to E19, wherein the risk of the subject having at least one progression event is reduced.

E20a. The method of any one of E4a, E4b, E4d to E7, or E20, wherein the risk is reduced by at least 5%, at least 10%, at least 15%, at least 20%, at least 25%, at least 30%, or at least 35%.

E20b. The method of any one of E4a, E4b, E4d to E7, E20, or E20a, wherein the risk reduction is in comparison to a subject with PPMS that is not administered fenebrutinib or a pharmaceutically acceptable salt thereof.

E20c. The method of any one of E4a, E4b, E4d to E7, or E20 to E20b, wherein the risk reduction is in comparison to a subject with PPMS who is administered an anti-CD20 antibody.

E21. A method of slowing the progression of PPMS in a subject in need thereof, comprising administering to the subject about 200 mg fenebrutinib twice per day, or an equivalent amount of a pharmaceutically acceptable salt thereof.

E22. A method of delaying the onset of at least one progression event in a subject with PPMS, the method comprising administering to the subject about 200 mg fenebrutinib twice daily, or an equivalent amount of a pharmaceutically acceptable salt thereof.

E23. A method of reducing the risk of a subject with PPMS having at least one progression event, the method comprising administering to the subject about 200 mg fenebrutinib twice daily, or an equivalent amount of a pharmaceutically acceptable salt thereof.

E24. The method of any one of E18 to E23, wherein the progression of PPMS is evaluated using the MSIS-29, Neuro-QoL Upper Extremity, PROMIS-FatigueMS, MSWS-12, PGI-S, WPAI:MS, PGI-C, EQ-5D-5L, C-SSRS, 9-HPT, T25FWT, EDSS, SDMT, MRI, or NfL levels.

E25. The method of any one of E18 to E24, wherein the progression of PPMS is evaluated using the CDP12, the cCDP12, the CDP24, or the cCDP24.

E26. The method of E21, wherein the progression of PPMS comprises the subject experiencing at least one progression event.

E27. The method of any one of E19 to E26, wherein the at least one progression event is selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.

E28. The method of any one of E19, E20, or E22 to E27, wherein the at least one progression event comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points.

E29. The method of any one of E19, E20, or E22 to E28, wherein the progression event is confirmed at least 12 weeks after the initial progression.

E30. The method of any one of E19, E20, or E22 to E28, wherein the progression event is confirmed at least 24 weeks after the initial progression.

E31. The method of any one of E18 to E30, wherein the progression is slowed, or the onset is delayed, or the risk is decreased, in comparison to a subject with PPMS that is not administered fenebrutinib or a pharmaceutically acceptable salt thereof.

E32. The method of any one of E18 to E31, wherein the progression is slowed, or the onset is delayed, or the risk is decreased in comparison to a subject that is administered an anti-CD20 antibody.

E33. The method of any one of E1 to E32, wherein progression of PPMS in the subject is slowed, or the onset of at least one progression event in the subject is delayed, or the risk of having at least one progression event in the subject is decreased by at least 5%, at least 10%, at least 15%, at least 20%, at least 25%, at least 30%, or at least 35%.

E34. A method of reducing disability in a subject with PPMS, the method comprising administering to the subject about 200 mg fenebrutinib twice per day, or an equivalent amount of a pharmaceutically acceptable salt thereof.

E35. The method of E34, wherein reducing disability comprises:
reducing the psychological impact of MS;
increasing upper limb function;
increasing walking ability;
decreasing fatigue;
improving work status; or
decreasing global impression of MS severity;
or any combinations thereof.

E36. The method of any one of E1 to E35, wherein the subject has a reduction in one or more symptoms of PPMS after beginning treatment with fenebrutinib, or a pharmaceutically acceptable salt thereof.

E37. The method of any one of E1 to E36, wherein one or more physical impacts of multiple sclerosis on the subject is decreased.

E38. A method of slowing the progression of PPMS in a subject in need thereof, comprising administering to the subject about 200 mg fenebrutinib twice per day, or an equivalent amount of a pharmaceutically acceptable salt thereof; wherein progression of PPMS comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.
   and wherein the progression event is confirmed at least 24 weeks after the initial progression.

E39. The method of E38, wherein the progression event is confirmed at least 12 weeks after the initial progression.

E40. The method of E38 or E39, wherein the at least one progression event comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points.

E41. The method of any one of E38 to E40, wherein the progression is slowed in comparison to a subject with PPMS that is not administered fenebrutinib or a pharmaceutically acceptable salt thereof.

E42. The method of any one of E38 to E41, wherein the progression is slowed in comparison to a subject that is administered an anti-CD20 antibody.

E43. The method of any one of E38 to E42, wherein the progression is slowed by at least 5%, at least 10%, at least 15%, at least 20%, at least 25%, at least 30%, or at least 35%.

E44. The method of any one of E1 to E43, wherein the method further comprises the step of measuring one or more clinical or laboratory endpoints in the subject in order to evaluate the efficacy of treating PPMS.

E45. The method of E44, wherein the one or more clinical or laboratory endpoints are selected from the group consisting of the subject's MSIS-29, Neuro-QoL Upper Extremity, PROMIS-FatigueMS, MSWS-12, PGI-S, WPAI:MS, PGI-C, EQ-5D-5L, C-SSRS, 9-HPT, T25FWT, EDSS, SDMT, MRI, or NfL levels.

E46. The method of E44 or E45, wherein the clinical or laboratory endpoint is measured 2 weeks, 6 weeks, 12 weeks, 18 weeks, 24 weeks, 36 weeks, 48 weeks, 60 weeks, 72 weeks, 84 weeks, 96 weeks, 108 weeks, or 120 weeks, or any combinations thereof, after beginning administration of fenebrutinib, or a pharmaceutically acceptable salt thereof.

E47. The method of any one of E44 to E46, wherein the clinical or laboratory endpoint is measured 120 weeks after beginning administration of fenebrutinib, or a pharmaceutically acceptable salt thereof.

E48. The method of any one of E1 to E47, wherein the development of one or more new MS-related brain lesion types in the subject is evaluated after the subject begins administration of fenebrutinib or a pharmaceutically acceptable salt thereof, wherein the one or more lesion types is selected from the group consisting of new gadolinium-enhancing lesions on a T1-weighted MRI (T1Gd+), new/enlarging T2-weighted lesions detected by MRI, or new T1-hypointense lesions detected by MRI.

E49. The method of E48, wherein the development of one or more new lesions in the subject is reduced as compared to a subject with PPMS that is not administered fenebrutinib or a pharmaceutically acceptable salt thereof, or a subject that is administered an anti-CD20 antibody, or a combination thereof.

E50. The method of any one of E2 to E47, wherein the period of evaluation is 120 weeks after beginning administration of fenebrutinib or a pharmaceutically acceptable salt thereof.

E51. The method of any one of E1 to E50, wherein the fenebrutinib or pharmaceutically acceptable salt thereof is administered orally.

E52. The method of any one of E1 to E51 wherein the fenebrutinib or pharmaceutically acceptable salt thereof is administered in the form of one or more tablets or capsules.

E53. The method of any one of E1 to E52, wherein the fenebrutinib or pharmaceutically acceptable salt thereof is administered in the form of two tablets twice daily, each tablet comprising about 100 mg fenebrutinib or an equivalent amount of a pharmaceutically acceptable salt thereof.

E54. The method of any one of E1 to E53, wherein the free form of fenebrutinib is administered.

E55. A compound for use in a method of treating primary progressive multiple sclerosis (PPMS) in a subject in need thereof, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

E56. The compound for use of E55, wherein disability progression in the subject is evaluated.

E57. The compound for use of E56, wherein disability progression is evaluated using the Expanded Disability Status Scale (EDSS), the 9-Hole Peg Test (9-HPT), or the Timed 25-Foot Walk Test (T25FWT), or any combinations thereof.

E58. The compound for use of E56 or E57, wherein the onset of composite 12-week confirmed disability progression (cCDP12) is evaluated, wherein onset of the cCDP12 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-1P; and
(c) increase from baseline of at least 20% in T25FWT.
and wherein the progression event is confirmed at least 12 weeks after the initial progression.

E58a. A compound for use in reducing the risk of experiencing cCDP12 in a subject with PPMS, comprising administering to the subject about 200 mg fenebrutinib twice daily, or an equivalent amount of a pharmaceutically acceptable salt thereof.

E58b. The compound for use of E58a, wherein cCDP12 comprises the first occurrence of a progression event in the subject after beginning of administration of fenebrutinib or a pharmaceutically acceptable salt thereof, wherein the progression event is confirmed at least 12 weeks after the initial disability progression.

E58c. A compound for use in reducing time to onset of cCDP12 in a subject with PPMS, comprising administering to the subject about 200 mg fenebrutinib twice daily, or an equivalent amount of a pharmaceutically acceptable salt thereof, wherein time to onset of cCDP12 comprises the period from before beginning administration of fenebrutinib or a pharmaceutically acceptable salt thereof to the first occurrence of a progression event, wherein the progression event is confirmed at least 12 weeks after the initial disability progression.

E58d. The compound for use of E58a or E58b, wherein the progression event is one of:
(a) an increase from baseline in Expanded Disability Status Scale (EDSS) score of ≥1.0 point in a subject with a baseline EDSS score of ≤5.5 or an increase of ≥0.5 points in a subject with a baseline EDSS score of >5.5 (confirmed disability progression [CDP];
(b) ≥20% increase from baseline in the Timed 25-Foot Walk Test (T25FWT); or
(c) ≥20% increase from baseline in time to complete the 9-Hole Peg Test (9-HPT).

E59. The compound for use of any one of E56 to E58d, wherein the method further comprises evaluating the onset of 12-week confirmed disability progression (CDP12) in the subject, wherein the onset of CDP12 comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points; and wherein the progression of EDSS is confirmed at least 12 weeks after the initial progression.

E60. The compound for use of any one of E56 to E59, wherein the method further comprises evaluating the onset of composite 24-week confirmed disability progression (cCDP24), wherein the onset of cCDP24 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.
and wherein the progression event is confirmed at least 24 weeks after the initial progression.

E61. The compound for use of any one of E56 to E60, wherein the method further comprises evaluating the onset of 24-week confirmed disability progression (CDP24) in the subject, wherein the onset of CDP24 comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points; and wherein the progression of EDSS is confirmed at least 24 weeks after the initial progression.

E62. The compound for use of any one of E55 to E61, wherein time to a progression event is increased, wherein the progression event is:
an increase from baseline in EDSS score of at least 1.0 point in a subject with a baseline EDSS score of less than or equal to 5.5 points; or
an increase from baseline in EDSS score of at least 0.5 point in a subject with a baseline EDSS score of greater than 5.5 points.

E63. The compound for use of any one of E55 to E62, wherein time to a progression event in the subject is increased, wherein the progression event is an increase from baseline of at least 20% in time to complete the 9-HPT.

E64. The compound for use of any one of E55 to E63, wherein time to a progression event in the subject is increased, wherein the progression event is an increase from baseline of at least 20% in T25FWT.

E65. The compound for use of any one of E55 to E64, wherein time to onset of CDP12 is increased.

E66. The compound for use of any one of E55 to E65, wherein time to onset of cCDP12 is increased.

E67. The compound for use of any one of E55 to E66, wherein time to onset of CDP24 is increased.

E68. The compound for use of any one of E55 to E67, wherein time to onset of cCDP24 is increased.

E69. The compound for use of any one of E62 to E68, wherein the increase is in comparison to a subject with PPMS who is not administered fenebrutinib or a pharmaceutically acceptable salt thereof.

E70. The compound for use of any one of E62 to E69, wherein the increase is in comparison to a subject with PPMS who is administered an anti-CD20 antibody.

E71. The compound for use of any one of E62 to E70, wherein the increase is at least 5%, at least 10%, at least 15%, at least 20%, at least 25%, at least 30%, or at least 35%.

E72. The compound for use of any one of E55 to E71, wherein the progression of PPMS in the subject is slowed.

E73. The compound for use of any one of E55 to E72, wherein the onset of at least one progression event is delayed.

E74. The compound for use of any one of E55 to E73, wherein the risk of the subject having at least one progression event is reduced.

E74a. The compound for use of any one of E58a, E58b, E58d to E61, or E74, wherein the risk is reduced by at least 5%, at least 10%, at least 15%, at least 20%, at least 25%, at least 30%, or at least 35%.

E74b. The compound for use of any one of E58a, E58b, E58d to E61, E74, or E74a, wherein the risk reduction is in comparison to a subject with PPMS that is not administered fenebrutinib or a pharmaceutically acceptable salt thereof.

E74c. The compound for use of any one of E58a, E58b, E58d to E61, or E74 to E74b, wherein the risk reduction is in comparison to a subject with PPMS who is administered an anti-CD20 antibody.

E75. A compound for use in a method of slowing the progression of PPMS in a subject in need thereof, wherein the compound is fenebrutinib or a pharmaceutically acceptable salt thereof, and wherein the method comprises administering to the subject about 200 mg fenebrutinib twice per day, or an equivalent amount of a pharmaceutically acceptable salt thereof.

E76. A compound for use in a method of delaying the onset of at least one progression event in a subject with PPMS, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

E77. A compound for use in a method of reducing the risk of a subject with PPMS having at least one progression event, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

E78. The compound for use of any one of E72 to E77, wherein the progression of PPMS is evaluated using the MSIS-29, Neuro-QoL Upper Extremity, PROMIS-FatigueMS, MSWS-12, PGI-S, WPAI:MS, PGI-C, EQ-5D-5L, C-SSRS, 9-HPT, T25FWT, EDSS, SDMT, MRI, or NfL levels.

E79. The compound for use of any one of E72 to E78, wherein the progression of PPMS is evaluated using the CDP12, the cCDP12, the CDP24, or the cCDP24.

E80. The compound for use of E79, wherein the progression of PPMS comprises at least one progression event.

E81. The compound for use of any one of E73 to E80, wherein the at least one progression event is selected from the group consisting of:
(a) an increase from baseline in EDSS score of at least 1.0 point in a subject with a baseline EDSS score of less than or equal to 5.5 points, or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.

E82. The compound for use of any one of E73 to E81, wherein the at least one progression event comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points.

E83. The compound for use of any one of E76 to E82, wherein the progression event is confirmed at least 12 weeks after the initial progression.

E84. The compound for use of any one of E76 to E82, wherein the progression event is confirmed at least 24 weeks after the initial progression.

E85. The compound for use of any one of E76 to E84, wherein the progression is slowed, or the onset is delayed, or the risk is decreased, in comparison to a subject with PPMS that is not administered fenebrutinib or a pharmaceutically acceptable salt thereof.

E86. The compound for use of any one of E76 to E85, wherein the progression is slowed, or the onset is delayed, or the risk is decreased in comparison to a subject that is administered an anti-CD20 antibody.

E87. The compound for use of any one of E55 to E86, wherein the progression of PPMS in the subject is slowed, or the onset of at least one progression event in the subject is delayed, or the risk of having at least one progression event in the subject is decreased by at least 5%, at least 10%, at least 15%, at least 20%, at least 25%, at least 30%, or at least 35%.

E88. A compound for use in a method of reducing disability in a subject with PPMS, wherein the compound is fenebrutinib or a pharmaceutically acceptable salt thereof, and wherein the method comprises administering to the subject about 200 mg fenebrutinib twice per day, or an equivalent amount of a pharmaceutically acceptable salt thereof.

E89. The compound for use of E88, wherein reducing disability comprises:
reducing the psychological impact of MS;
increasing upper limb function;
increasing walking ability;
decreasing fatigue,
improving work status; or
decreasing global impression of MS severity;
or any combinations thereof.

E90. The compound for use of any one of E55 to E89, wherein the subject has a reduction in one or more symptoms of PPMS after beginning treatment with fenebrutinib, or a pharmaceutically acceptable salt thereof.

E91. The compound for use of any one of E55 to E90, wherein one or more physical impacts of multiple sclerosis on the subject is decreased.

E92. A compound for use in a method of slowing the progression of PPMS in a subject in need thereof, wherein the compound is fenebrutinib or a pharmaceutically acceptable salt thereof, and wherein the method comprises administering to the subject about 200 mg fenebrutinib twice per day, or an equivalent amount of a pharmaceutically acceptable salt thereof; wherein progression of PPMS comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.
and wherein the progression event is confirmed at least 24 weeks after the initial progression.

E93. The compound for use of E92, wherein the progression event is confirmed at least 12 weeks after the initial progression.

E94. The compound for use of E92 or E93, wherein the at least one progression event comprises an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points.

E95. The compound for use of any one of E92 to E94, wherein the progression is slowed in comparison to a subject with PPMS that is not administered fenebrutinib or a pharmaceutically acceptable salt thereof.

E96. The compound for use of any one of E92 to E95, wherein the progression is slowed in comparison to a subject that is administered an anti-CD20 antibody.

E97. The compound for use of any one of E92 to E96, wherein the progression is slowed by at least 5%, at least 10%, at least 15%, at least 20%, at least 25%, at least 30%, or at least 35%.

E98. The compound for use of any one of E55 to E97, wherein the method further comprises the step of measuring one or more clinical or laboratory endpoints in the subject in order to evaluate the efficacy of treating PPMS.

E99. The compound for use of E98, wherein the one or more clinical or laboratory endpoints are selected from the group consisting of the subject's MSIS-29, Neuro-QoL Upper Extremity, PROMIS-FatigueMS, MSWS-12, PGI-S, WPAI:MS, PGI-C, EQ-5D-5L, C-SSRS, 9-HPT, T25FWT, EDSS, SDMT, MRI, or NfL levels.

E100. The compound for use of E98 or E99, wherein the clinical or laboratory endpoint is measured 2 weeks, 6 weeks, 12 weeks, 18 weeks, 24 weeks, 36 weeks, 48 weeks, 60 weeks, 72 weeks, 84 weeks, 96 weeks, 108 weeks, or 120 weeks, or any combinations thereof, after beginning administration of fenebrutinib, or a pharmaceutically acceptable salt thereof.

E101. The compound for use of any one of E98 to E100, wherein the clinical or laboratory endpoint is measured 120 weeks after beginning administration of fenebrutinib, or a pharmaceutically acceptable salt thereof.

E102. The compound for use of any one of E55 to E101, wherein the development of one or more new MS-related brain lesion types in the subject is evaluated after the subject begins administration of fenebrutinib or a pharmaceutically acceptable salt thereof, wherein the one or more lesion types is selected from the group consisting of new gadolinium-enhancing lesions on a T1-weighted MRI (T1Gd+), new/enlarging T2-weighted lesions detected by MRI, or new T1-hypointense lesions detected by MRI.

E103. The compound for use of E102, wherein the development of one or more new lesions in the subject is reduced as compared to a subject with PPMS that is not administered fenebrutinib or a pharmaceutically acceptable salt thereof, or a subject that is administered an anti-CD20 antibody, or a combination thereof.

E104. The compound for use of any one of E56 to E101, wherein the period of evaluation is 120 weeks after beginning administration of fenebrutinib or a pharmaceutically acceptable salt thereof.

E105. The compound for use of any one of E55 to E104, wherein the fenebrutinib or pharmaceutically acceptable salt thereof is administered orally.

E106. The compound for use of any one of E55 to E105, wherein the fenebrutinib or pharmaceutically acceptable salt thereof is administered in the form of one or more tablets or capsules.

E107. The compound for use of any one of E55 to E106, wherein the fenebrutinib or pharmaceutically acceptable salt thereof is administered in the form of two tablets twice daily, each tablet comprising about 100 mg fenebrutinib or an equivalent amount of a pharmaceutically acceptable salt thereof.

E108. The compound for use of any one of E54 to E107, wherein compound is the free form of fenebrutinib.

E109. Further provided herein is a compound for use in the manufacture of a medicament for any of the methods of E1 to E54, wherein the compound is fenebrutinib or a pharmaceutically acceptable salt thereof.

E110. The method of any one of E1 to E53, or the compound for use of any one of E54 to E108, wherein the subject with PPMS has had progressive disease from the onset, and has been in a progressive stage for at least 12 months prior to beginning administration of fenebrutinib or a pharmaceutically acceptable salt thereof.

E111. The method of any one of E1 to E53 or E110, or the compound for use of any one of E54 to E108 or E110, wherein prior to beginning administration of fenebrutinib or a pharmaceutically acceptable salt thereof, the subject has at least two of
(a) one or more T2-hyperintense lesions characteristic of MS in one or more of the periventricular, cortical or juxtacortical, or infratentorial the following brain regions;
(b) two or more T2-hyperintense lesions in the spinal cord; and
(c) the presence of cerebrospinal fluid-specific oligoclonal bands.

E112. The method of any one of E1 to E53, E110, or E111, or the compound for use of any one of E54 to E108, E110, or E111, wherein the subject has an EDSS score from 3.0 to 6.5 prior to beginning administration of fenebrutinib or a pharmaceutically acceptable salt thereof.

E113. The method of any one of E1 to E53 or E110 to E112, or the compound for use of any one of E54 to E108 or E110 to E112, wherein the subject with PPMS does not have one or more of:
estimated glomerular filtration rate (eGFR)<60 mL/min/1.73 m²;
ALT or AST>2×ULN;
total bilirubin greater than 1.5×ULN;
hemoglobin<9.5 g/dL;
platelet count<100×10⁹/L; or
abnormalities in one or more of the hepatic synthetic function tests PT, INR, PTT, or albumin.

E114. Further provided herein is a method of any one of E1 to E54 or E110 to E113, or compound for use of any one of E55 to E108 or E110 to E113, wherein the subject is not concomitantly administered a strong CYP3A4 inhibitor while being administered about 200 mg fenebrutinib twice per day, or an equivalent amount of a pharmaceutically acceptable salt thereof.

E115. The method or compound for use of E114, wherein the strong CYP3A4 inhibitor is boceprevir, cobicistat, clarithromycin, danoprevir/ritonavir, elvitegravir/ritonavir, indinavir/ritonavir, itraconazole, idelalisib, ketoconazole, lopinavir/ritonavir, nefazodone, nelfinavir, posaconazole, ritonavir, saquinavir, telaprevir, telithromycin, or voriconazole.

E116. Further provided herein is a method of any one of E1 to E54 or E110 to E114, or compound for use of any one of E55 to E108 or E110 to E114, wherein the subject is not concomitantly administered a strong CYP3A4 inducer while being administered about 200 mg fenebrutinib twice per day, or an equivalent amount of a pharmaceutically acceptable salt thereof.

E117. The method or compound for use of E116, wherein the strong CYP3A4 inducer is apalutamide, carbamazepine, enzalutamide, mitotane, phenytoin, rifampin, or hyperforin (St. John's Wort).

E118. Further provided herein is a method of any one of E1 to E54 or E110 to E117, or compound for use of any one of E55 to E108 or E110 to E114, wherein the subject is not concomitantly administered a moderate CYP3A4 inducer while being administered about 200 mg fenebrutinib twice per day, or an equivalent amount of a pharmaceutically acceptable salt thereof.

E119. The method or compound for use of E118, wherein the moderate CYP3A4 inducer is bosentan, dexamethasone, efavirenz, etravirine, phenobarbital, primidone, phenobarbital, or rifabutin.

E120. Further provided herein is a method of any one of E1 to E54 or E110 to E119, or compound for use of any one of E55 to E108 or E110 to E119, wherein the subject is not concomitantly administered a CYP3A4 substrate with a narrow therapeutic window while being administered about 200 mg fenebrutinib twice per day, or an equivalent amount of a pharmaceutically acceptable salt thereof.

E121. The method or compound for use of E120, wherein the CYP3A4 substrate with a narrow therapeutic window is alfentanil, astemizole, cyclosporine, cisapride, dihydroergotamine, ergotamine, everolimus, fentanyl, pimozide, quinidine, sirolimus, terfenadine, or tacrolimus.

### BRIEF DESCRIPTION OF THE DRAWINGS

FIG. 1 depicts the comparative kinase selectivity of fenebrutinib compared to three other BTK inhibitors.

### DETAILED DESCRIPTION

Provided herein are methods and uses of fenebrutinib, or a pharmaceutically acceptable salt of fenebrutinib, for treating Primary Progressive Multiple Sclerosis (PPMS).

Fenebrutinib is a compound of the formula:

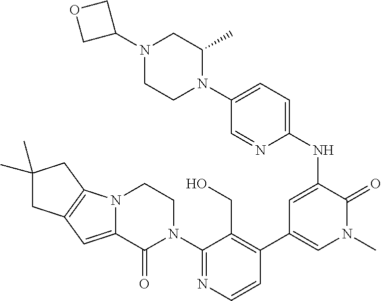 [SMILES: C[C@H]1CN(C2COC2)CCN1c1ccc(Nc2cc(-c3ccnc(N4CCn5c(cc6c5CC(C)(C)C6)C4=O)c3CO)cn(C)c2=O)nc1]

and is also known by the following names:
GDC-0853,
(6²S)-2³-(hydroxymethyl)-1⁷,1⁷,3⁷, 6²-tetramethyl-1³,1⁴,1⁷,1⁸-tetrahydro-4-aza-1(2)-cyclopenta[4,5]pyrrolo[1,2-a]pyrazina-6(1,4)-piperazina-2(2,4),3(3,5),5(2,5)-tripyridina-7(3)-oxetanaheptaphane-1¹(1⁶H),3⁶(3¹H)-dione; and
(S)-2-3′-(hydroxymethyl)-1-methyl-5-((5-(2-methyl-4-(oxetan-3-yl)piperazin-1-yl)pyridin-2-yl)amino)-6-oxo-1,6-dihydro-[3,4′-bipyridin]-2′-yl)-7,7-dimethyl-2,3,4,6,7,8-hexahydro-1H-cyclopenta[4,5]pyrrolo[1,2-a]pyrazin-1-one.

Additional names for the same compound may be known, for example using different chemical naming schemes. The R enantiomer of the compound is: (R)-2-(3′-(hydroxymethyl)-1-methyl-5-4(5-(2-methyl-4-(oxetan-3-yl)piperazin-1-yl)pyridin-2-yl)amino)-6-oxo-1,6-dihydro-[3,4′-bipyridin]-2′-yl)-7,7-dimethyl-2,3,4,6,7,8-hexahydro-1H-cyclopenta[4,5]pyrrolo[1,2-a]pyrazin-1-one.

Fenebrutinib is a highly selective, orally administered, reversible inhibitor of BTK. U.S. Pat. No. 8,716,274, which is hereby incorporated by reference in its entirety, discloses classes of heteroaryl pyridine and aza-pyridone compounds useful for inhibiting Btk, including fenebrutinib. WO 2017/148837, which is hereby incorporated by reference in its entirety, discloses solid forms and formulations of fenebrutinib and pharmaceutically acceptable salts thereof.

### I. Definitions

It is to be understood that the terminology used herein is for the purpose of describing particular embodiments only, and is not intended to be limiting.

As used in this specification, including the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se. In some embodiments, the term “about” refers to a range of plus or minus 10% for the respective value. In some embodiments, the term “about” refers to a range of plus or minus 5% for the respective value. In some embodiments, the term “about” refers to a range of plus or minus 2% for the respective value. In some embodiments, the term “about” refers to a range of plus or minus 1% for the respective value.

It is understood that aspects and embodiments of the disclosure described herein include “comprising,” “consisting,” and “consisting essentially of” aspects and embodiments.

The term “pharmaceutical formulation” refers to a preparation which is in such form as to permit the biological activity of the active ingredient to be effective, and which contains no additional components which are unacceptably toxic to a subject to which the formulation would be administered. In some embodiments, such formulations are sterile. “Pharmaceutically acceptable” excipients (vehicles, additives) are those which can reasonably be administered to a subject mammal to provide an effective dose of the active ingredient employed.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. In some embodiments, two or more of such effects are achieved. In some embodiments, an individual is successfully “treated” if one or more symptoms associated with their disease or disorder is diminished; the disease is made more tolerable to the subject; the rate of degeneration or decline, or rate of disease or disorder development is slowed or stopped; the progression of the disease or disorder is slowed or stopped; or the final point of degeneration is less debilitating. For example, an individual is successfully “treated” if one or more symptoms associated with cancer are mitigated or eliminated, including, but are not limited to, decreasing symptoms resulting from the disease, increasing the quality of life of those suffering from the disease, decreasing the dose of other medications required to treat the disease, and/or prolonging survival of individuals. Treatment of certain diseases or disorders may in some embodiments include, but is not limited to, specific clinical or other endpoints such as those described in the Examples provided herein.

Some embodiments described herein refer to providing a dose of fenebrutinib, or an equivalent amount of a pharmaceutically acceptable salt thereof. It would be clear to one of skill in the art how to calculate a corresponding amount of a pharmaceutical salt form of fenebrutinib, taking into account the difference in molecular weight between the free form of fenebrutinib and a salt form. For example, in some embodiments provided herein, a subject is administered about 400 mg daily of fenebrutinib (as two, 200 mg doses), or a pharmaceutically acceptable salt thereof. If a pharmaceutically acceptable salt form is administered in such embodiments, due to the salt form having a higher molecular weight than the free form of fenebrutinib, the total weight of the pharmaceutically acceptable salt of fenebrutinib administered daily is greater than 400 mg, but corresponds to about 400 mg of the free form of fenebrutinib.

A “subject” for purposes of treatment refers to any animal classified as a mammal, including humans, domestic and farm animals, and zoo, sports, or pet animals, such as dogs, horses, cats, cows, etc. In some embodiments of the methods provided herein, the subject is human. In some embodiments, the subject is a patient.

“Prior to beginning administration” may include, for example, on the same day as, but before the actual administration of, the first dose of fenebrutinib or pharmaceutically acceptable salt thereof is administered; or within one week prior to the first dose; or within two weeks prior to the first dose; or within three weeks prior to the first dose; or within four weeks prior to the first dose; or within five weeks prior to the first dose; or within six weeks prior to the first dose; or within greater than six weeks prior to the first dose; or between 1 and 28 days prior to the first dose; or within 0 to 28 days prior to the first dose. In certain embodiments, this period of time may also be referred to as “baseline”. Thus, in some embodiments, baseline may include within one week prior to administering the first dose of fenebrutinib or a pharmaceutically acceptable salt thereof, including the same day just prior to administration. In other embodiments, baseline includes within one month, or within 0 to 28 days, or within six week prior to the first dose of fenebrutinib or a pharmaceutically acceptable salt thereof.

The term “biomarker” as used herein refers to an indicator, e.g., predictive, diagnostic, and/or prognostic, which can be detected in a sample. The biomarker may serve as an indicator of a particular subtype of a disease or disorder (e.g., multiple sclerosis) characterized by certain, molecular, pathological, histological, and/or clinical features. In some embodiments, a biomarker is a gene. Biomarkers may include, but are not limited to, polynucleotides (e.g., DNA, and/or RNA), polypeptides, polypeptide and polynucleotide modifications (e.g. posttranslational modifications), carbohydrates, and/or glycolipid-based molecular markers. The “amount” or “level” of a biomarker associated with an increased clinical benefit to an individual is a detectable level in a biological sample. These can be measured by methods known to one skilled in the art and also disclosed herein. The expression level or amount of biomarker assessed can, in some embodiments, be used to determine the response to the treatment. In certain embodiments, the expression level or amount of one or more biomarkers is associated with a certain response to treatment.

The term “sample.” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumor lysates, and tissue culture medium, tissue extracts such as homogenized tissue, tumor tissue, cellular extracts, and combinations thereof. In some embodiments, the sample is a blood sample. In other embodiments, the sample is cerebrospinal fluid (CSF).

By “tissue sample” or “cell sample” is meant a collection of similar cells obtained from a tissue of a subject or individual. The source of the tissue or cell sample may be solid tissue as from a fresh, frozen and/or preserved organ, biopsy, and/or aspirate; blood or any blood constituents such as plasma; bodily fluids such as cerebrospinal fluid, amniotic fluid, peritoneal fluid, or interstitial fluid; cells from any time in gestation or development of the subject. The tissue or cell sample may also be primary or cultured cells or cell lines. Optionally, the tissue or cell sample is obtained from a disease tissue/organ. The tissue or cell sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue. In another embodiment, a reference sample is obtained from an untreated tissue and/or cell of the body of the same subject or individual, such as, for example, a sample taken from the subject or individual prior to beginning a particular treatment (e.g., prior to beginning treatment with fenebrutinib or a pharmaceutically acceptable salt thereof). In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

The “Expanded Disability Status Scale” (EDSS) is a ClinRO measure for quantifying changes in the disability level of a subject with MS over time. The EDSS is based on a standard neurological examination, incorporating functional systems (visual, brainstem, pyramidal, cerebellar, sensory, bowel and bladder, and cerebral [or mental]) that are rated and then scored as a functional system score (FSS), and ambulation, which is scored as ambulation score. Each FSS is an ordinal clinical rating scale ranging from 0 to 5 or 6, and an ambulation score that is rated from 0 to 12. These ratings may then be used in conjunction with observations, as well as information, concerning ambulation and use of assistive devices to determine the total EDSS score. The EDSS is a disability scale that ranges in 0.5-point steps from 0 (normal) to 10.0 (death) (Kurtzke 1983; Kappos 2011). In some embodiments of the methods provided herein, the item sexual dysfunction and fatigue are not included in the EDSS score.

The “9-Hole Peg Test” (9-HPT) is a quantitative measure of upper extremity (arm and hand) function (Goodkin et al. 1988; Fischer et al. 2001). The test device consists of a container with nine pegs and a block containing nine empty holes. The subject is to pick up each of the nine pegs one at a time and as quickly as possible place them in the nine holes. Once all the pegs are in the holes, the subject is to remove them again one at a time as quickly as possible and replace them into the container. The total time to complete the task is recorded. Both the dominant and non-dominant hands are tested twice (two successfully completed trials of the dominant hand, followed immediately by two successfully completed trials of the non-dominant hand). The two trials for each hand are averaged, converted to the reciprocals of the mean times for each hand, and the two reciprocals are averaged. The 9-HPT may be administered, for example, as described in the Multiple Sclerosis Functional Composite (MSFC) Administration and Scoring Manual (Fischer et al., 2001). A meaningful change in upper extremity function may, for example, be indicated by a 20% worsening from baseline of the averaged 9-HPT times.

The “Timed 25-Foot Walk Test” (T25FWT) is a quantitative measure of mobility and leg function, based on a timed 25-foot walk. The subject is directed to start at one end of a clearly marked 25-foot course and is instructed to walk 25 feet as quickly and safely as possible, and how long it takes the subject to go from start of the walk to the end of the 25 feet is timed. In some embodiments, the task is administered immediately again by having the subject walk back the same distance, and the time for both completed trials averaged to produce the score for the T25FWT. Subjects may use assistive devices (e.g., cane or wheelchair) when performing the task. The T25FWT may be administered, for example, as described in the MSFC Administration and Scoring Manual (Fischer et al., 2001). A clinically meaningful change in mobility and leg function may, for example, be indicated by a 20% worsening from baseline of the averaged T25FWT time.

The “Symbol Digit Modalities Test” (SDMT) is a test used to evaluate the presence of cognitive impairment and/or changes in cognitive functioning over time and in response to treatment. The SDMT may be particularly sensitive to slowed processing of information that is commonly seen in MS (Benedict et al. 2017). The SDMT comprises a substitution task. Using a reference key, the subject has 90 seconds to pair specific numbers with given geometric figures. Responses may be collected orally, and the number of correct responses is considered the SDMT score. A clinically meaningful change in cognitive processing may, for example, be indicated by a decrease by 4 points on the SDMT score from baseline.

The “Columbia-Suicide Severity Rating Scale” (C-SSRS) is a tool used to assess the lifetime suicidality of a subject, and may be used to track suicidal events through treatment or a portion thereof. The structured interview prompts recollection of suicidal ideation, including the intensity of the ideation, behavior, and attempts with actual/potential lethality. A “baseline” C-SSRS may include, for example, C-SSRS collected prior to beginning administration of fenebrutinib or a pharmaceutically acceptable salt thereof. Such score may be compared, for example, to subsequent C-SSRS collected after beginning administration of fenebrutinib or a pharmaceutically acceptable salt thereof. Comparisons between different evaluation periods (which may, for example, occur during visits with a clinician) may be described, in some embodiments, as “since last visit” C-SSRS.

The “EQ-5D-5L” is a validated self-reported health status questionnaire that can used to calculate a health status utility score for use in health economic analyses (EuroQol Group 1990; Brooks 1996; Herdman et al. 2011; Janssen et al. 2013). There are two components to the EQ-5D-5L: a five-item health state profile that assesses mobility, self-care, usual activities, pain/discomfort, and anxiety/depression, as well as a visual analog scale (VAS) that measures health state. The EQ-5D-5L is designed to capture a subject's current health status. Published weighting systems may allow for creation of a single composite score of the subject's health status.

The “Multiple Sclerosis Impact Scale-29 Version 2” (MSIS-29, Version 2) is a 29-item subject-reported measure of the physical and psychological impacts of MS (Hobart et al. 2001). Subjects are asked to rate how much their functioning and well-being has been impacted over the past 14 days on a 4-point scale, from “Not at all” (1) to “Extremely” (4). The physical score is the sum of items 1-20, which is then transformed to a 0-100 scale. The psychological score is the sum of items 21-29, transformed to a 0-100 scale. Higher scores may indicate a greater impact of MS. A clinically meaningful impact is indicated by a change of at least 7.5 points on the physical scale in Version 1 of the MSIS-29. In Version 2 of the MSIS-29, this level of change may also indicate a meaningful impact.

The “Multiple Sclerosis Walking Scale, 12-Item” (MSWS-12) is a 12-item self-report measure of the impact of MS on the individual's ability to walk during the past 2 weeks. Each item is scored on a 5-point Likert scale, and total scores are converted to a 0-100 scale with higher scores indicating greater impact of MS on walking ability.

The “Quality of Life in Neurological Disorders, Upper Extremity” (fine motor skills and activities of daily living; Neuro-QoL, Upper Extremity) is a 20-item questionnaire used to assess upper limb function, which involves subjects with MS through each stage of its development (Gershon et al. 2012). Items include assessments of dressing, cooking, eating, cleaning, and writing from which the subject uses a 5-point Likert scale to rate his or her performance ranging from “without any difficulty” (5) to “unable to do” (1). Item scores are summed, multiplied by 20 and divided by 20 minus the number of any unanswered items. Scores range from 20-100, where a higher score indicates better upper limb function. In accordance with the NINDS User Manual (2015), scores can be calculated as long as at least 50% of the items have been answered.

The “PROMIS-FatigueMS” is an 8-item scale developed as a measure of fatigue for subjects with MS (Cook et al. 2012) with a recall period of the previous 7 days. It comprises a 5-point Likert-type scale that produces a score between 1 and 5 for each scored question. The total raw score is the sum of the values of each scored question. The total raw score ranges from 8-40. Scores can also be transformed to a PROMIS T-score where the mean is 50 and a standard deviation of 10. T-scores range from 34.7-81.3. A higher score is associated with worse fatigue.

The “Patient Global Impression of Change” (PGI-C) is a single-item assessment of a subject's impression of his or her change in MS symptoms compared with a point 6 months previous. Subjects respond on a 7-point Likert scale from “very much better” (1) to “very much worse” (7). The PGI-C is used as an anchor for determining what is a clinically meaningful change in the MSIS-29.

The “Patient Global Impression of Severity” (PGI-S) is a single-item assessment of a subject's impression of the severity of his or her MS symptoms from the past 7 days. A subject responds on a 5-point Likert scale from “none” (1) to “very severe” (5). The PGI-S is used as an anchor for determining what is a clinically meaningful change in the MSIS-29.

The “Work Productivity and Activity Impairment: Multiple Sclerosis” (WPAI:MS) is a 6-item scale. A subject estimates the amount of time that their work and daily activities were affected by their MS over the previous 7 days (Reilly et al. 1993). The WPAI:MS assesses absenteeism as well as “presenteeism,” which accounts for the time when a subject was present for work or activities, but believed their health had a negative effect on their ability to perform at the usual level. A higher score represents a greater impairment in productivity.

“Confirmed Disability Progression” (CDP) refers to an increase in the subject's EDSS score that is sustained over a particular time period. This may be evaluated, for example, by calculating the subject's EDSS score, determining that the score is increased over a previous score (such as a baseline score, which may be a score taken before the subject began administration of fenebrutinib or a pharmaceutically acceptable salt thereof), and then confirming the score is still increased after a specified period of time has elapsed from the initial increase (e.g., by reevaluating the subject and recalculating it again). For example, a 12-week confirmed disability progression (CDP12) refers to an EDSS score that remains increased at least 12 weeks after the initial increase (e.g., as confirmed by recalculating the EDSS score at least 12 weeks after the initial increase). A 24-week confirmed disability progression (CDP24) refers to an EDSS score remains increased at least 24 weeks after the initial increase (e.g., as confirmed by recalculating the EDSS score at least 24 weeks after the initial increase). The initial increase may be compared to a baseline EDSS score (such as prior to beginning administration with fenebrutinib or a pharmaceutically acceptable salt thereof), or may be compared to a prior EDSS score that had remained stable over time, such as over 12, 24, 36, 48, or 60 weeks. In some embodiments, a CDP refers to an increase of ≥1.0 point from the baseline EDSS score in a subject with a baseline EDSS score of ≤5.5 points, or an increase of ≥0.5 point from the baseline EDSS score in a subject with a baseline EDSS score of >5.5 points. Time to onset of a CDP (e.g., time to onset of CDP12 or CDP24) refers to the time period from when the prior EDSS score was established (for example, a baseline EDSS score from before beginning administration of fenebrutinib or a pharmaceutically acceptable salt thereof) until the sustained increase of EDSS score is observed.

“Composite Confirmed Disability Progression” (cCDP) is a composite measure of disability progression using a combination of EDSS, 9-HPT, and T25FWT. It evaluates the progression of subject's disability over a particular time period as determined by the first occurrence of a progression event. A progression event may include any one of the following: a CDP (e.g., increase of ≥1.0 point from the baseline EDSS score in a subject with a baseline EDSS score of ≤5.5 points, or an increase of ≥0.5 point from the baseline EDSS score in a subject with a baseline EDSS score of >5.5 points); an increase of ≥20% from baseline in time to complete the 9-Hole Peg Test (9-HPT); or an increase of ≥20% from baseline in the Timed 25-Foot Walk Test (T25FWT); wherein the occurrence of the progression event is confirmed at after a specified period of time has elapsed from the initial occurrence. For example, a composite 12-week confirmed disability progression (cCDP12) refers to the occurrence of at least one progression event at an initial time point, and the same progression event is confirmed at least 12 weeks later (e.g., by re-evaluating the subject using the same test). A composite 24-week confirmed disability progression (cCDP12) refers to the occurrence of at least one progression event at an initial time period, and same progression event is confirmed at least 24 weeks later. Time to onset of a cCDP (e.g., time to onset of cCDP12 or cCDP24) refers to the time period from when the prior evaluation scores were established (for example, baseline scores before beginning administration of fenebrutinib or a pharmaceutically acceptable salt thereof) until the initial progression event is observed. Without wishing to be bound by theory, compared with endpoints based exclusively on the Expanded Disability Status Scale (EDSS), which emphasizes lower limb function, the cCDP12 requires at least one of the following: 1) an increase in EDSS score of ≥1.0 point from a baseline (BL) score of ≤5.5 points, or ≥0.5 point increase from a BL score of >5.5 points (Confirmed Disability Progression); 2) a 20% increase from BL in time to complete the 9-Hole Peg Test; 3) a 20% increase from BL in the Timed 25-Foot Walk Test. Thus, the cCDP12 is a more sensitive assessment of disability, especially at early disease stages. The use of the cCDP12 as a primary outcome may provide a clearer, more complete picture of disability progression or improvement than the EDSS alone.

### II. Methods of Treatment

Without wishing to be bound by theory, BTK inhibition results in a decrease in activation and proliferation of B cells, which may explain its effects on the inflammatory pathways related to MS disease activity. BTK inhibition also has direct effects on myeloid lineage cells. As a result, there is a potential for BTK inhibition to affect microglia that are associated with the pathophysiology of MS disease progression independent of relapse.

Provided herein are methods of treating PPMS in a subject in need thereof, by administering to the subject a daily dose of about 200 mg fenebrutinib twice daily, or a corresponding amount of a pharmaceutically acceptable salt thereof, for a total daily dose of about 400 mg fenebrutinib, or an equivalent amount of a pharmaceutically acceptable salt thereof. Further provided is a compound for use in a method of treating PPMS in a subject in need thereof, wherein the compound is a fenebrutinib or a pharmaceutically acceptable salt thereof, and wherein the method comprises administering to the subject a twice daily dose of about 200 mg fenebrutinib, or a corresponding amount of a pharmaceutically acceptable salt thereof. In further embodiments, provided herein is a compound for use in the manufacture of a medicament for the treatment of PPMS in a subject in need thereof, wherein the compound is a fenebrutinib or a pharmaceutically acceptable salt thereof, and wherein the treatment comprises administering to the subject a twice daily dose of about 200 mg fenebrutinib, or a corresponding amount of a pharmaceutically acceptable salt thereof. In some embodiments, the treatment of PPMS is evaluated using the Expanded Disability Status Scale (EDSS), the 9-Hole Peg Test (9-HPT), or the Timed 25-Foot Walk Test (T25FWT), or any combinations thereof. In some embodiments, the treatment of PPMS is evaluated based the time to onset of confirmed disability progression (e.g., 12-week or 24-week CDP), or based on the time to onset of a composite confirmed disability progression (e.g., 12-week or 24-week cCDP). For example, in some embodiments, treating a subject with PPMS by administering about 200 mg of fenebrutinib twice daily, or an equivalent amount of a pharmaceutically acceptable salt thereof, results in a delay in worsening of the EDSS (e.g., increase of 0.5, 1.0, 1.5, or more points compared to baseline), a delay in the worsening of the 9-HPT time (e.g., by over 20% compared to baseline), a delay in the worsening of the T25FWT time (e.g., by over 20% compared to baseline), delaying to onset of CDP12, delaying to onset of CDP24, delaying to the onset of cCDP12, delaying the onset of cCDP24, delaying the onset of at least one progression event, reducing the risk of having at least one progression event, or decreasing disability in a subject with PPMS. In other embodiments, the treatment of PPMS is evaluated based on MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels. For example, in some embodiments, treating a subject with PPMS comprises delaying the progression of PPMS, wherein the progression is evaluated based on MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels; or the onset of at least one progression event, which may be described by CDP12, cCDP12, CDP24, or cCDP24. In some embodiments, treating PPMS comprises delaying progression of PPMS. In certain embodiments, treating PPMS comprises delaying the onset of at least one progression event in the subject. In some embodiments, treating PPMS comprises reducing the risk of the subject experiencing at least one progression event. In certain embodiments, treating PPMS comprises delaying progression, or delaying the onset of at least one progression event, by at least 5%, at least 10%, at least 15%, at least 20%, at least 25%, at least 30%, or at least 35% (e.g., as evaluated using T25FWT time, or 9-HPT time, or EDSS score, or CDP12, or cCDP12, or CDP24, or cCDP24 etc.). In certain embodiments, treating PPMS comprises delaying progression, or delaying the onset of at least one progression event, by at least 5%, at least 10%, at least 15%, at least 20%, at least 25%, at least 30/6, or at least 35% as compared to another subject with PPMS (e.g., a comparative subject), wherein the other subject is not administered fenebrutinib or a pharmaceutically acceptable salt thereof. In some embodiments, the delay is at least 5%. In some embodiments, the delay is at least 10%. In some embodiments, the delay is at least 15%. In some embodiments, the delay is at least 20%. In some embodiments, the delay is at least 25%. In some embodiments, the delay is at least 30%. In some embodiments, the delay is at least 35%. In some embodiments, the other subject is administered an anti-CD20 antibody (such as a CD20-directed cytolytic antibody). In still further embodiments, treating PPMS comprises reducing the risk the subject has at least one progression event by at least 5%, at least 10%, at least 15%, at least 20%, at least 25%, at least 30%, or at least 35%. In certain embodiments, the risk is reduced over a period of time, for example reducing the risk of having at least one progression event over 12 weeks, 18 weeks, 24 weeks, 36 weeks, 48 weeks, 60 weeks, 72 weeks, 84 weeks, 96 weeks, 108 weeks, or 120 weeks. In some embodiments, the risk is reduced as compared to another subject with PPMS (e.g., a comparative subject) who is not administered fenebrutinib, or a pharmaceutically acceptable salt thereof, and who is optionally administered an anti-CD20 antibody. In some embodiments, the other subject is administered an anti-CD20 antibody (such as a CD20-directed cytolytic antibody). In some embodiments, the risk is reduced by at least 5%. In some embodiments, the risk is reduced by at least 10%. In some embodiments, the risk is reduced by at least 15%. In some embodiments, the risk is reduced by at least 20%. In some embodiments, the risk is reduced by at least 25%. In some embodiments, the risk is reduced by at least 25%. In some embodiments, the risk is reduced by at least 30%. In some embodiments, the risk is reduced by at least 35%. In certain embodiments, treating PPMS comprises an improvement of at least 5%, at least 10%, at least 15%, at least 20%, at least 25%, or at least 30% in a metric of PPMS (e.g., in T25FWT time, or 9-HPT time, or EDSS score, etc.), as compared to the same metric evaluated in the same subject prior to beginning administration of fenebrutinib or a pharmaceutically acceptable salt thereof. In some embodiments, the improvement is compared to the same metric evaluated in the same subject within 1 week, or within 0 to 28 days, or within 6 weeks prior to beginning administration of fenebrutinib or a pharmaceutically acceptable salt thereof. In other embodiments, treating PPMS comprises an improvement of at least 5%, at least 10%, at least 15%, at least 20%, at least 25%, at least 30%, or at least 40% in a metric of PPMS (e.g., in T25FWT time, or 9-HPT time, or EDSS score, etc.), as compared to the same metric evaluated in another subject with PPMS, wherein the other subject is not administered fenebrutinib or a pharmaceutically acceptable salt thereof. In some embodiments, the improvement is at least 5%. In some embodiments, the improvement is at least 10%. In some embodiments, the improvement is at least 15%. In some embodiments, the improvement is at least 20%. In some embodiments, the improvement is at least 25%. In some embodiments, the improvement is at least 30%. In some embodiments, the improvement is at least 35%. In some embodiments, the other subject is administered an anti-CD20 antibody (such as a CD20-directed cytolytic antibody).

Further provided is a method of treating (e.g., slowing) progression of PPMS in a subject in need thereof, by administering to the subject a twice daily dose of about 200 mg fenebrutinib, or a corresponding amount of a pharmaceutically acceptable salt thereof, for a total daily dose of about 400 mg fenebrutinib, or an equivalent amount of a pharmaceutically acceptable salt thereof. Thus, provided herein are methods of treating (e.g. slowing) the progression of PPMS in a subject in need thereof, by administering to the subject about 200 mg fenebrutinib twice daily, or a corresponding amount of a pharmaceutically acceptable salt thereof. Further provided is a compound for use in a method of slowing the progression of PPMS in a subject in need thereof, wherein the compound is a fenebrutinib or a pharmaceutically acceptable salt thereof, and wherein the method comprises administering to the subject a twice daily dose of about 200 mg fenebrutinib, or a corresponding amount of a pharmaceutically acceptable salt thereof. In further embodiments, provided herein is a compound for use in the manufacture of a medicament for use in a method of treating (e.g., slowing) the progression of PPMS in a subject in need thereof, wherein the compound is a fenebrutinib or a pharmaceutically acceptable salt thereof, and wherein the method comprises administering to the subject a twice daily dose of about 200 mg fenebrutinib, or a corresponding amount of a pharmaceutically acceptable salt thereof. In some embodiments, the progression of PPMS is evaluated using the Expanded Disability Status Scale (EDSS), the 9-Hole Peg Test (9-HPT), or the Timed 25-Foot Walk Test (T25FWT), or any combinations thereof. In some embodiments, the progression of PPMS is evaluated based the time to onset of confirmed disability progression (e.g., 12-week or 24-week CDP), or based on the time to onset of a composite confirmed disability progression (e.g., 12-week or 24-week cCDP). In certain embodiments, the progression of PPMS is slowed at least 5%, at least 10%, at least 15%, at least 20%, at least 25%, at least 30% or at least 35%. In some embodiments, the progression is slowed at least 5%. In some embodiments, the progression is slowed at least 10%. In some embodiments, the progression is slowed at least 15%. In some embodiments, the progression is slowed at least 20%. In some embodiments, the progression is slowed at least 25%. In some embodiments, the progression is slowed at least 30%. In some embodiments, the progression is slowed at least 35%. In some embodiments, progression is slowed as measured by the onset of cCDP12 (e.g., by increasing the time to onset of cCDP12) or by the risk of cCDP12 (e.g., reducing the risk of experiencing cCDP12 during a period of time). In some embodiments, the progression of PPMS is slowed relative to another subject with PPMS (e.g., a comparator subject), wherein the other subject is not administered fenebrutinib or a pharmaceutically acceptable salt thereof. In some embodiments, the other subject is administered an anti-CD20 antibody (such as a CD20-directed cytolytic antibody), and is not administered a BTK inhibitor (such as fenebrutinib or a pharmaceutically acceptable salt thereof). In certain embodiments, the total evaluation time period is 12 weeks, 18 weeks, 24 weeks, 36 weeks, 48 weeks, 60 weeks, 72 weeks, 84 weeks, 96 weeks, 108 weeks, or 120 weeks. In certain embodiments, the total evaluation time period is at least 120 weeks, e.g., PPMS progression is slowed by at least 5%, at least 10%, at least 15%, at least 20%, at least 25%, at least 30%, or at least 35%, as evaluated over 120 weeks, when compared to another subject with PPMS who is not administered fenebrutinib or a pharmaceutically acceptable salt thereof and is optionally administered a CD20-directed cytolytic antibody. In some embodiments, the other subject is administered an anti-CD20 antibody (such as a CD20-directed cytolytic antibody).

In still further embodiments, provided herein is a method of decreasing disability in a subject with PPMS, comprising administering to the subject about 200 mg fenebrutinib twice per day, or an equivalent amount of a pharmaceutically acceptable salt thereof, for a total daily dose of about 400 mg fenebrutinib, or an equivalent amount of a pharmaceutically acceptable salt thereof. In some embodiments, provided is a compound for use in a method of decreasing disability in a subject with PPMS, wherein the compound is fenebrutinib or a pharmaceutically acceptable salt thereof, and the method comprises administering to the subject about 200 mg fenebrutinib twice per day, or an equivalent amount of a pharmaceutically acceptable salt thereof. In further embodiments, provided herein is a compound for use in the manufacture of a medicament for use in a method of decreasing disability in a subject with PPMS, wherein the compound is fenebrutinib or a pharmaceutically acceptable salt thereof, and wherein the method comprises administering to the subject a daily dose of about 400 mg fenebrutinib, or a corresponding amount of a pharmaceutically acceptable salt thereof. Decreasing disability may comprise reducing the psychological impact of MS; increasing upper limb function; increasing walking ability; decreasing fatigue; improving work status; or decreasing global impression of MS severity; or any combinations thereof. Decreasing disability may further include decreasing one or more symptoms of PPMS, or decreasing one or more physical impacts of PPMS on the subject. The decrease in disability (including, for example, one or more symptoms or physical impacts, or other aspects as described herein) may be evaluated as described herein, such as using MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels. In some embodiments, one or more of 9-HPT, T25FWT, or EDSS is used. In some embodiments, decreasing disability comprises a subject that can complete the T25FWT and/or 9-HPT more quickly, or a decrease in the EDSS score (e.g., closer to “normal”). In certain embodiments, a decrease in disability comprises an improvement in one or more metrics of PPMS, such as one evaluated using MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels. In certain embodiments, the improvement is at least 5%, at least 10%, at least 15%, at least 20%, at least 25%, at least 30%, or at least 35% in at least one metric of PPMS (e.g., in T25FWT time, or 9-HPT time, or EDSS score), as compared to the same metric evaluated in the same subject prior to beginning administration of fenebrutinib or a pharmaceutically acceptable salt thereof. In some embodiments, two, three, four, five or more metrics are improved, wherein each improvement level is independent (e.g., one metric improves by at least 10%, another metric improves by at least 20%). In some embodiments, the improvement is at least 5%. In some embodiments, the improvement is at least 10%. In some embodiments, the improvement is at least 15%. In some embodiments, the improvement is at least 20%. In some embodiments, the improvement is at least 25%. In some embodiments, the improvement is at least 30%. In some embodiments, the improvement is at least 35%. In some embodiments, the improvement is compared to the same metric evaluated in the same subject within 1 week, or within 0 to 28 days, or within 6 weeks prior to beginning administration of fenebrutinib or a pharmaceutically acceptable salt thereof.

In still further embodiments, provided is a method of delaying the onset of at least one progression event in a subject with PPMS, the method comprising administering to the subject a twice daily dose of about 200 mg fenebrutinib, or a pharmaceutically acceptable salt thereof, for a total daily dose of about 400 mg fenebrutinib, or an equivalent amount of a pharmaceutically acceptable salt thereof. Further provided is a compound for use in a method of delaying the onset of at least one progression event in a subject with PPMS, wherein the compound is fenebrutinib or a pharmaceutically acceptable salt thereof, and wherein the method comprises administering to the subject about 200 mg fenebrutinib twice daily or an equivalent amount of a pharmaceutically acceptable salt thereof, for a total daily dose of about 400 mg fenebrutinib, or an equivalent amount of a pharmaceutically acceptable salt thereof. In yet further embodiments, provided is a compound for use in manufacture of a medicament for a method of delaying the onset of at least one progression event in a subject with PPMS, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof. A progression event may include, for example, an increase from baseline in the time needed to complete the 9-HPT, or an increase from baseline in the time needed to complete the T25FWT, or an increase from baseline of the EDSS score. In some embodiments, the increase from baseline in time needed to complete the 9-HPT is an increase of at least 20% (e.g., may be 20%, 25%, 30%, 35% etc.). In some embodiments, the increase from baseline in the time needed to complete the T25FWT is an increase of at least 20% (e.g., may be 20%, 25%, 30%, 35% etc.). In still further embodiments, the increase from baseline of the EDSS score is an increase of at least 1.0 wherein the baseline is less than or equal to 5.5 points; or an increase of at least 0.5 point in a subject with a baseline score of greater than 5.5 points. In certain embodiments, the progression event is confirmed a certain time period after the initial progression, such as at least 12 weeks, or at least 24 weeks. In certain embodiments, the baseline used in determining a progression event is the same metric (e.g., T25FWT, 9-HPT, EDSS, or combinations thereof) evaluated in the same subject within 1 week, or within 0 to 28 days, or within 6 weeks prior to beginning administration of fenebrutinib or a pharmaceutically acceptable salt thereof. In certain embodiments, the methods, compounds for use, or use of a compound in the manufacture of a medicament, delays the onset of at least one progression event by at least 5%, at least 10%, at least 15%, at least 20%, at least 25%, at least 30%, or at least 35%. In some embodiments, the delay is at least 5%. In some embodiments, the delay is at least 10%. In some embodiments, the delay is at least 15%. In some embodiments, the delay is at least 20%. In some embodiments, the delay is at least 25%. In some embodiments, the delay is at least 25%. In some embodiments, the delay is at least 30%. In some embodiments, the delay is at least 35%. In some embodiments, the time to onset is delayed relative to another subject with PPMS (e.g., a comparator subject), wherein the other subject is not administered a BTK inhibitor (such as fenebrutinib or a pharmaceutically acceptable salt thereof). In some embodiments, the other subject is administered an anti-CD20 antibody (such as a CD20-directed cytolytic antibody), and is not administered a BTK inhibitor (such as fenebrutinib or a pharmaceutically acceptable salt thereof). In certain embodiments, the total evaluation time period is 12 weeks, 18 weeks, 24 weeks, 36 weeks, 48 weeks, 60 weeks, 72 weeks, 84 weeks, 96 weeks, 108 weeks, or 120 weeks. In certain embodiments, the total evaluation time period is at least 120 weeks, e.g., the time period until onset of at least one progression event is increased by at least 5%, at least 10%, at least 15%, at least 20%, at least 25%, at least 30%, or at least 35%, as evaluated over 120 weeks, when compared to another subject with PPMS who is not administered a BTK inhibitor (such as fenebrutinib or a pharmaceutically acceptable salt thereof) and is optionally administered an anti-CD20 antibody (such as a CD20-directed cytolytic antibody). In some embodiments, calculating the delay in onset of at least one progression event may comprise, for example, calculating the additional time until the onset of a progression event in subject administered fenebrutinib or a pharmaceutically acceptable salt thereof, as compared to a subject not administered fenebrutinib or a pharmaceutically acceptable salt thereof (and optionally administered an anti-CD20 antibody).

Further provided herein is a method of reducing the risk of a subject with PPMS having at least one progression event, the method comprising administering to the subject about 200 mg of fenebrutinib twice daily, or an equivalent amount of a pharmaceutically acceptable salt thereof, for a total daily dose of about 400 mg fenebrutinib, or an equivalent amount of a pharmaceutically acceptable salt thereof. Further provided is a compound for use in a method of reducing the risk of a subject with PPMS having at least one progression event, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof. In yet further embodiments, provided is a compound for use in manufacture of a medicament for reducing the risk of a subject with PPMS having at least one progression event, wherein the compound is fenebrutinib or a pharmaceutically acceptable salt thereof, and wherein the subject is administered about 200 mg fenebrutinib twice daily, or an equivalent amount of a pharmaceutically acceptable salt thereof. A progression event may include, for example, an increase from baseline in the time needed to complete the 9-HPT, or an increase from baseline in the time needed to complete the T25FWT, or an increase from baseline of the EDSS score. In some embodiments, the increase from baseline in time needed to complete the 9-HPT is an increase of at least 20% (e.g., may be 20%, 25%, 30%, etc.). In some embodiments, the increase from baseline in the time needed to complete the T25FWT is an increase of at least 20% (e.g., may be 20%, 25%, 30%, etc.). In still further embodiments, the increase from baseline of the EDSS score is an increase of at least 1.0 (e.g., may be 1.0, 1.5, 2.0, etc.) wherein the baseline is less than or equal to 5.5 points; or an increase of at least 0.5 point (e.g., may be 0.5, 1.0, 1.5, etc.) in a subject with a baseline score of greater than 5.5 points. In certain embodiments, the progression event is confirmed a certain time period after the initial progression, such as at least 12 weeks, or at least 24 weeks (e.g., as CDP12, cCDP12, CDP24, or cCDP24). In certain embodiments, the baseline used in determining a progression event is the same metric (e.g., T25FWT, 9-HPT, EDSS, or combinations thereof) evaluated in the same subject within 1 week, or within 0 to 28 days, or within 6 weeks prior to beginning administration of fenebrutinib or a pharmaceutically acceptable salt thereof. In certain embodiments, the methods, compounds for use, or use of a compound in the manufacture of a medicament, reduces the risk of the subject with PPMS having at least one progression event by at least 5%, at least 10%, at least 15%, at least 20%, at least 25%, at least 30%, or at least 35%. In some embodiments, the risk is reduced at least 5%. In some embodiments, the risk is reduced at least 10%. In some embodiments, the risk is reduced at least 15%. In some embodiments, the risk is reduced at least 20%. In some embodiments, the risk is reduced at least 25%. In some embodiments, the risk is reduced at least 30%. In some embodiments, the risk is reduced at least 35%. In some embodiments, the risk of having at least one progression event comprises reducing the risk of experiencing cCDP12, or reducing the risk of worsening according to EDSS. In some embodiments, the reduced risk of having at least one progression event is reduced relative to another subject with PPMS, wherein the other subject is not administered a BTK inhibitor (such as fenebrutinib, or a pharmaceutically acceptable salt thereof). In some embodiments, the other subject is administered an anti-CD20 antibody (such as a CD20-directed cytolytic antibody), and is not administered a BTK inhibitor (such as fenebrutinib or a pharmaceutically acceptable salt thereof). In certain embodiments, the total evaluation time period is 12 weeks, 18 weeks, 24 weeks, 36 weeks, 48 weeks, 60 weeks, 72 weeks, 84 weeks, 96 weeks, 108 weeks, or 120 weeks. In certain embodiments, the total evaluation time period is at least 120 weeks, e.g., a subject with PPMS administered fenebrutinib or a pharmaceutically acceptable salt thereof has at least 5%, at least 10%, at least 15%, at least 20%, at least 25%, at least 30%, or at least 35% lower risk of having at least one progression event over 120 weeks, when compared to another subject with PPMS who is not administered fenebrutinib or a pharmaceutically acceptable salt thereof and is optionally administered a CD20-directed cytolytic antibody. Such reduced risk of having at least one progression event may be calculated, for example, by calculating the rate of progression events in one or more subject with PPMS administered fenebrutinib or a pharmaceutically acceptable salt thereof (e.g., over 60 weeks, or over 120 weeks) and comparing that rate to the rate of progression events in one or more subject with PPMS not administered fenebrutinib or a pharmaceutically acceptable salt thereof over and optionally administered an anti-CD20 antibody (such as a CD20-directed cytolytic antibody).

Further provided herein are methods of increasing mobility in a subject in need thereof, wherein the subject has PPMS, comprising administering to the subject about 200 mg fenebrutinib twice daily, or a corresponding amount of a pharmaceutically acceptable salt thereof, for a total daily dose of about 400 mg fenebrutinib, or an equivalent amount of a pharmaceutically acceptable salt thereof. Thus, provided herein are methods of increasing mobility in a subject in need thereof, wherein the subject has PPMS, by administering to the subject about 200 mg fenebrutinib twice daily, or a corresponding amount of a pharmaceutically acceptable salt thereof. Increased mobility in subject may include, for example, increased ability to walk, increased ability to run, increased ability to climb up and/or down stairs, increased ability to stand, improved balance when walking or standing, increased distance a subject is able to walk, decreased effort needed to walk, decreased reliance on supports when walking indoors and/or outdoors (e.g., walking stick, leaning on furniture, walking frame, etc.), decreased amount of concentration required to walk, or increase in the evenness/smoothness of walking, or any combination of the foregoing. In some embodiments, one, two, or more of these aspects of mobility is improved, while one or more is not improved. For example, increasing mobility in a subject may comprise increased ability to walk, while one or more other components of mobility is not improved. Such increased mobility may, for example, be assessed using a subject questionnaire. In some embodiments, increased mobility, or one or more components of increased mobility as described herein, may be evaluated using the MSWS-12. In some embodiments, an increase in mobility is evaluated compared to the mobility (e.g., as evaluated using MSWS-12) of the subject prior to beginning administration of fenebrutinib, or a pharmaceutically acceptable salt thereof.

In some embodiments as provided herein, the progression of PPMS may be evaluated by one or more clinical or laboratory endpoints selected from the group consisting of MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels. Thus, for example, in some embodiments the progression of PPMS is evaluated by one or more of EDSS, T25FWT, or 9-HPT. Further, in some embodiments the progression of PPMS is evaluated by a sustained increase in one or more PPMS symptoms or signs, such as an increase that is sustained over at least 12 weeks (e.g., confirmed to still be increased at least 12 weeks after the initial increase observed), or at least 24 weeks (e.g., confirmed to still be increased at least 24 weeks after the initial increase observed). In certain embodiments, the progression of PPMS is evaluated by a cCDP or CDP, such as cCDP-12, CDP-12, cCDP24, or CDP24, or any combinations thereof. In some embodiments, progression of PPMS is evaluated by cCDP12. In certain embodiments, progression of PPMS is evaluated by EDSS. In certain embodiments, the risk of experiencing cCDP12 is decreased, or time to onset of cCDP12 is increased, in combination with reducing the risk of experiencing CDP12, or in combination with increasing the time to onset of CDP12. In certain embodiments, the risk of experiencing cCDP12 is decreased, or time to onset of cCDP12 is increased, in combination with reducing the risk of experiencing cCDP24, or in combination with increasing the time to onset of cCDP24. In still further embodiments, the risk of experiencing cCDP12 is decreased, in combination with both: reducing the risk of experiencing CDP12, and reducing the risk of experiencing cCDP24. In still further embodiments, time to onset of cCDP12 is increased in combination with both: increasing the time to onset of CDP12, and increasing the time to onset of cCDP24.

In some embodiments described herein, the response of a subject administered fenebrutinib or a pharmaceutically acceptable salt thereof may be compared to another subject who is administered an antibody to CD20 (e.g., an anti-CD20 antibody). As used herein an anti-CD20 antibody may include antibodies which bind to CD20, a cell surface antigen present on pre-B and mature B lymphocytes. In some embodiments, the antibody is a humanized monoclonal antibody directed against CD20-expressing B-cells. In certain embodiments, binding of the anti-CD20 antibody to the cell surface of B lymphocytes may result in antibody-dependent cellular cytolysis, and complement mediated lysis. In certain embodiments, the anti-CD20 antibody is a CD20-directed cytolytic antibody. Examples of such antibodies may include, for example, ocrelizumab. Ocrelizumab is a recombinant humanized, glycosylated, monoclonal IgG1 antibody that selectively targets and depletes CD20-expressing B cells.

In some embodiments of the methods, compounds for use, or use of a compound as described herein, about 200 mg fenebrutinib, or a corresponding amount of a pharmaceutically acceptable salt thereof, is adminstered twice daily to a subject with PPMS, wherein the subject with PPMS has had progressive disease from the onset, and a progressive stage for at least 12 months prior to beginning administration of fenebrutinib or a pharmaceutically acceptable salt thereof. In some embodiments, the subject with PPMS has one or more T2-hyperintense lesions in one or more of the periventricular, cortical or juxtacortical, or infratentorial brain regions; two or more T2-hyperintense lesions in the spinal cord; or the presence of cerebrospinal fluid-specific oligoclonal bands. In certain embodiments, the subject with PPMS has at least two of one or more T2-hyperintense lesions in one or more of the periventricular, cortical or juxtacortical, or infratentorial brain regions; two or more T2-hyperintense lesions in the spinal cord; or the presence of cerebrospinal fluid-specific oligoclonal bands. In still further embodiments, the subject with PPMS has had progressive disease from the onset, and a progressive stage for at least 12 months prior to beginning administration of fenebrutinib or a pharmaceutically acceptable salt thereof; and has at least two of: one or more T2-hyperintense lesions in one or more of the periventricular, cortical or juxtacortical, or infratentorial brain regions; two or more T2-hyperintense lesions in the spinal cord; or the presence of cerebrospinal fluid-specific oligoclonal bands. T2-hyperintense lesions may be evaluated, for example, by MRI. The presence of cerebrospinal fluid-specific oligoclonal bands may be evaluated, for example, by lumbar puncture. In further embodiments, the subject with PPMS may have an EDSS score from between 3.0 to 6.5 prior to beginning administration of fenebrutinib, or a pharmaceutically acceptable salt thereof. In some embodiments, the subject with PPMS is neurologically stable for at least 30 days prior to beginning administration of fenebrutinib, or a pharmaceutically acceptable salt thereof. In some such embodiments, the method, compound for use, or use of a compound, is for treating PPMS; treating (e.g. slowing) progression of PPMS); decreasing disability; delaying the onset of at least one progression event; reducing the risk of having at least one progression event; increasing mobility; or increasing time to onset of cCDP12, in a subject with PPMS in need thereof; and comprises administering to the subject in need thereof 200 mg of fenebrutinib, or an equivalent amount of a pharmaceutically acceptable salt thereof, twice daily.

In some embodiments of the methods, compounds for use, or use of a compound as described herein, about 200 mg fenebrutinib, or a corresponding amount of a pharmaceutically acceptable salt thereof, is adminstered twice daily to a subject with PPMS, wherein the subject with PPMS does not have progressive multifocal leukoencephalopathy, or does not have a history of progressive multifocal leukoencephalopathy. In certain embodiments, the subject with PPMS does not have a history of cancer within 10 years prior to beginning administration of fenebrutinib, or a pharmaceutically acceptable salt thereof. In certain embodiments, the subject with PPMS has not had a hematological malignancy or solid tumor within 10 years prior to beginning administration of fenebrutinib, or a pharmaceutically acceptable salt thereof. In certain embodiments, the subject with PPMS is not in an immunocompromised state. An immunocompromised state may include, for example, a CD4 count<250/uL or an ANC<1.5×10³/uL or serum IgG<4.6 g/L. In still further embodiments, the subject with PPMS does not have any other neurological disorders. Such other neurological disorders may include, for example, a history of an ischemic cerebrovascular disorder (e.g., stroke, transient ischemic attack, spontaneous intracranial hemorrhage, or traumatic intracranial hemorrhage) or ischemia of the spinal cord; history or known presence of a CNS or spinal cord tumor (e.g., meningioma or glioma); history or known presence of potential metabolic causes of myelopathy (e.g., untreated vitamin B12 deficiency); history or known presence of infectious causes of myelopathy (e.g., syphilis, Lyme disease, HTLV-1, herpes zoster myelopathy); history of genetically inherited progressive CNS degenerative disorder (e.g., hereditary paraparesis, mitochondrial myopathy, encephalopathy, lactic acidosis, stroke syndrome); neuromyelitis optica spectrum disorder; history or known presence of systemic autoimmune disorders potentially causing progressive neurological disease (e.g., lupus, anti-phospholipid antibody syndrome, Sjögren syndrome, Behçet disease); history or known presence of sarcoidosis; or history of severe, clinically significant brain or spinal cord trauma (e.g., cerebral contusion, spinal cord compression). In some embodiments, the subject with PPMS does not have evidence of clinically significant cardiovascular (including arrhythmias or QTc prolongation), psychiatric, pulmonary, renal, hepatic (including Gilbert's Syndrome), endocrine (including uncontrolled diabetes, non-gallstone pancreatitis, or chronic pancreatitis), metabolic, or gastrointestinal (GI) disease. In some embodiments, the subject with PPMS does not have heart disease. In certain embodiments, the subject with PPMS does not have congestive heart failure. Congestive heart failure may be evaluated, for example, using the New York Heart Association criteria. In certain embodiments, the subject with PPMS does not meet the Class III and Class IV criteria for congestive heart failure as described by the New York Heart Association. In still further embodiments, the subject with PPMS does not have a history of ventricular dysrhythmias or risk factors for ventricular dysrhythmias such as long QT syndrome or other genetic risk factors (e.g., Brugada syndrome); structural heart disease, coronary heart disease (symptomatic or with ischemia demonstrated by diagnostic testing, prior coronary artery bypass grafting, or coronary lesions>70% diameter stenosis that have not been or cannot be re-vascularized); clinically significant electrolyte abnormalities (e.g., hypokalemia, hypomagnesemia, hypocalcemia); family history of sudden, unexplained death; or cardiac ion channel genetic mutations (e.g., congenital long QT syndrome). In certain embodiments, the subject with PPMS is not concomitantly administered systemic corticosteroids, or immunosuppressants while taking fenebrutinib or a pharmaceutically acceptable salt thereof. In some embodiments, the subject with PPMS has not been adminstered systemic corticosteroids within 4 weeks prior to beginning administration of fenebrutinib, or pharmaceutically acceptable salt thereof. In some embodiments, the subject with PPMS has not been administered IV Ig or plasmapheresis within 12 weeks prior to beginning administration of fenebrutinib, or pharmaceutically acceptable salt thereof. In certain embodiments, the subject with PPMS does not have abnormal hepatic synthetic function. In still further embodiments, the subject with PPMS, while being administered fenebrutinib or a pharmaceutically acceptable salt thereof, is not concomitantly administered any one or more of: a CYP3A4 inhibitor, such as a strong CYP3A4 inhibitor; or a CYP3A4 inducer, such as a strong or moderate CYP3A4 inducer, or a CYP3A4 substrate; or fingolimod, siponimod, or ozanimod; or natalizumab; or dimethyl fumarate, interferons, or glatiramer acetate; or an anti-CD20; or mycophenolate mofetil or methotrexate; or teriflunomide; or cladribine, mitoxantrone, daclizumab, alemtuzumab, or cyclophosphamide. In yet other embodiments, the subject with PPMS, prior to beginning administration of fenebrutinib or a pharmaceutically acceptable salt thereof, is not administered: a strong CYP3A4 inhibitor, or a strong or moderate CYP3A4 inducer, within 7 days or 5 drug elimination half-lives (whichever is longer); a CYP3A4 substrate with a narrow therapeutic window within 7 days or 5 drug elimination half-lives (whichever is longer); an anti-CD20 within 2 years; fingolimod, siponimod, or ozanimod within 8 weeks; natalizumab within 6 months, if natalizumab was administered for more than one year; dimethyl fumarate, interferons, or glatiramer acetate within 4 weeks; mycophenolate mofetil or methotrexate within 12 weeks; teriflunomide, unless teriflunomide plasma concentrations are <0.02 mg/L; or cladribine, mitoxantrone, daclizumab, alemtuzumab, or cyclophosphamide. In yet further embodiments, the subject with PPMS does not have one or more of: an estimated glomerular filtration rate (eGFR)<60 mL/min/1.73 m²; an ALT or AST>2×ULN; a total bilirubin greater than 1.5×ULN; a hemoglobin<9.5 g/dL; a platelet count<100×109/L; or a clinically significant abnormality in one or more hepatic synthetic function tests (such as PT, INR, PTT, or albumin). In certain embodiments, the subject with PPMS has an estimated glomerular filtration rate (eGFR)≥60 mL/min/1.73 m²; an ALT or AST<2×ULN; a total bilirubin less than 1.5×ULN; a hemoglobin≥9.5 g/dL; or a platelet count>100×109/L. In some such embodiments, the method, compound for use, or use of a compound, is for treating PPMS; treating (e.g. slowing) progression of PPMS); decreasing disability; delaying the onset of at least one progression event; reducing the risk of having at least one progression event; increasing mobility; or increasing time to onset of cCDP12, in a subject with PPMS in need thereof; and comprises administering to the subject in need thereof 200 mg of fenebrutinib, or an equivalent amount of a pharmaceutically acceptable salt thereof, twice daily.

### III. Pharmaceutical Compositions and Formulations

Also provided herein are pharmaceutical compositions and formulations comprising fenebrutinib, or a pharmaceutically acceptable salt thereof, for use in the methods of treatment described herein (e.g., treating PPMS, delaying the progression of PPMS, etc.). In some embodiments, the pharmaceutical compositions and formulations further comprise one or more pharmaceutically acceptable carriers. WO 2017/148837, which is hereby incorporated by reference in its entirety, discloses formulations and dosage forms comprising fenebrutinib and pharmaceutically acceptable salts thereof. In some embodiments, a formulation described in WO 2017/148837 is used to deliver fenebrutinib to a subject according to one or more of the methods provided herein.

Fenebrutinib, or a pharmaceutically acceptable salt thereof can be administered by any suitable means, including oral, parenteral, intrapulmonary, and intranasal, and, if desired for local treatment, intralesional administration. In certain embodiments, oral administration is used.

Pharmaceutically acceptable salts of fenebrutinib may be used in the methods herein. As used herein, the term “pharmaceutically acceptable salt” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

In some of the embodiments provided herein, an oral dose of fenebrutinib, or a pharmaceutically acceptable salt thereof, is administered as one or more tablets or capsules. For example, in some embodiments, about 200 mg of fenebrutinib, or an equivalent amount of a pharmaceutically acceptable salt thereof, is administered twice daily as one or more tablets, such as one, two, three, four, five, or six tablets administered twice daily. In other embodiments, about 200 mg of fenebrutinib, or an equivalent amount of a pharmaceutically acceptable salt thereof, is administered twice daily as one or more capsules, such as one, two, three, four, five, or six capsules administered twice daily. Such capsules or tablets may contain, in some embodiments, about 50 mg, about 75 mg, about 100 mg, about 125 mg, about 150 mg, about 175, mg, about 200 mg, or about 225 mg each of fenebrutinib, or an equivalent amount of a pharmaceutically acceptable salt thereof. For example, in certain embodiments, about 200 mg is administered twice daily to a subject in need thereof, wherein each 200 mg dose is administered as one capsule comprising about 200 mg fenebrutinib, or an equivalent amount of a pharmaceutically acceptable salt thereof; or each 200 mg dose is administered as two capsules comprising about 100 mg fenebrutinib, or an equivalent amount of a pharmaceutically acceptable salt thereof. In certain embodiments, about 200 mg of fenebrutinib is administered twice daily (e.g., about 400 mg total daily), wherein each 200 mg is administered as two capsules comprising about 100 mg fenebrutinib. In other embodiments, about 200 mg is administered twice daily to a subject in need thereof, wherein each 200 mg dose is administered as one tablet comprising about 200 mg fenebrutinib, or an equivalent amount of a pharmaceutically acceptable salt thereof; or each 200 mg dose is administered as two tablets comprising about 100 mg fenebrutinib, or an equivalent amount of a pharmaceutically acceptable salt thereof. In certain embodiments, about 200 mg of fenebrutinib is administered twice daily (e.g., about 400 mg total daily), wherein each 200 mg is administered as two tablets comprising about 100 mg fenebrutinib. Thus, in some embodiments, the daily dose of fenebrutinib is about 400 mg daily, such as from about 360 mg to about 440 mg daily, or an equivalent amount of a pharmaceutically acceptable salt of fenebrutinib. In certain embodiments, 400 mg of fenebrutinib is administered daily.

In further embodiments as provided herein, an article of manufacture or a kit is provided comprising fenebrutinib, or a pharmaceutically acceptable salt thereof, and a container. In certain embodiments, further include is a package insert comprising instructions for using fenebrutinib, or a pharmaceutically acceptable salt thereof. Suitable containers for kits include, for example, a bottle, a box, a blister pack, or a combinations thereof (e.g., a blister pack in a box). In some embodiments, the container holds the formulation and the label on, or associated with, the container may indicate directions for use. The article of manufacture or kit may further include other materials desirable from a commercial and user standpoint, including package inserts with instructions for use.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### ENUMERATED EMBODIMENTS

Embodiment 1. A method of treating primary progressive multiple sclerosis (PPMS) in a subject in need thereof, comprising administering to the subject about 200 mg fenebrutinib twice daily, or an equivalent amount of a pharmaceutically acceptable salt thereof.

Embodiment 2. The method of embodiment 1, further comprising evaluating disability progression in the subject, wherein disability progression is evaluated using the Expanded Disability Status Scale (EDSS), the 9-Hole Peg Test (9-HPT), or the Timed 25-Foot Walk Test (T25FWT), or any combinations thereof.

Embodiment 3. The method of embodiment 1 or 2, further comprising evaluating the onset of composite 12-week confirmed disability progression (cCDP12), wherein onset of the cCDP12 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.
and wherein the progression event is confirmed at least 12 weeks after the initial progression.

Embodiment 4. The method of any one of embodiments 1 to 3, wherein time to a progression event in the subject is increased, wherein the progression event is:
an increase from baseline in EDSS score of at least 1.0 point in a subject with a baseline EDSS score of less than or equal to 5.5 points; or
an increase from baseline in EDSS score of at least 0.5 point in a subject with a baseline EDSS score of greater than 5.5 points.

Embodiment 5. The method of any one of embodiments 1 to 4, wherein time to a progression event in the subject is increased, wherein the progression event is increase of at least 20% from baseline in time to complete the 9-HPT.

Embodiment 6. The method of any one of embodiments 1 to 5, wherein time to a progression event in the subject is increased, wherein the progression event is an increase of at least 20% from baseline in T25FWT.

Embodiment 7. The method of any one of embodiments 1 to 6, wherein time to onset of CDP12, cCDP12, CDP24, or cCDP24 is increased in comparison to a subject with PPMS who is not administered fenebrutinib or a pharmaceutically acceptable salt thereof.

Embodiment 8. The method of embodiment 7, wherein the subject with PPMS who is not administered fenebrutinib is administered an anti-CD20 antibody.

Embodiment 9. The method of any one of embodiments 4 to 8, wherein the time to a progression event or time to onset is increased at least 10%.

Embodiment 10. A method of slowing the progression of PPMS in a subject in need thereof, comprising administering to the subject about 200 mg fenebrutinib twice per day, or an equivalent amount of a pharmaceutically acceptable salt thereof.

Embodiment 11. The method of embodiment 10, wherein the progression of PPMS is evaluated using the MSTS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels.

Embodiment 12. The method of embodiment 10 or 11, wherein the progression of PPMS comprises at least one progression event.

Embodiment 13. A method of delaying the onset of at least one progression event in a subject with PPMS, the method comprising administering to the subject about 200 mg fenebrutinib twice daily, or an equivalent amount of a pharmaceutically acceptable salt thereof.

Embodiment 14. A method of reducing the risk of a subject with PPMS having at least one progression event, the method comprising administering to the subject about 200 mg fenebrutinib twice daily, or an equivalent amount of a pharmaceutically acceptable salt thereof.

Embodiment 15. The method of any one of embodiments 12 to 14, wherein the at least one progression event is selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.

Embodiment 16. The method of embodiment 15, wherein the progression event is confirmed at least 12 weeks after the initial progression.

Embodiment 17. The method of any one of embodiments 1 to 12, 15, or 16, wherein the progression of PPMS in the subject is slowed by at least 10% compared to another subject with PPMS who is not administered fenebrutinib or a pharmaceutically acceptable salt thereof and is administered an anti-CD20 antibody.

Embodiment 18. The method of any one of embodiments 13, 15, or 16, wherein the onset of at least one progression event is delayed by at least 10% compared to another subject with PPMS who is not administered fenebrutinib or a pharmaceutically acceptable salt thereof and is administered an anti-CD20 antibody.

Embodiment 19. The method of any one of embodiments 1 to 16, wherein the risk of the subject having at least one progression event is decreased by at least 10% compared to another subject with PPMS who is not administered fenebrutinib or a pharmaceutically acceptable salt thereof and is administered an anti-CD20 antibody.

Embodiment 20. A method of reducing disability in a subject with PPMS, the method comprising administering to the subject about 200 mg fenebrutinib twice per day, or an equivalent amount of a pharmaceutically acceptable salt thereof.

Embodiment 21. The method of embodiment 20, wherein reducing disability comprises:
reducing the psychological impact of MS;
increasing upper limb function;
increasing walking ability;
decreasing fatigue;
improving work status; or
decreasing global impression of MS severity;
or any combinations thereof.

Embodiment 22. The method of any one of embodiments 1 to 21, wherein the subject has a reduction in one or more symptoms of PPMS after beginning treatment with fenebrutinib, or a pharmaceutically acceptable salt thereof.

Embodiment 23. The method of any one of embodiments 1 to 22, wherein the method further comprises the step of measuring one or more clinical or laboratory endpoints in the subject in order to evaluate the efficacy of treating PPMS.

Embodiment 24. The method of embodiment 23, wherein the one or more clinical or laboratory endpoints are selected from the group consisting of the subject's MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels.

Embodiment 25. The method of any one of embodiments 1 to 24, wherein the fenebrutinib or pharmaceutically acceptable salt thereof is administered orally.

Embodiment 26. The method of any one of embodiments 1 to 25, wherein the fenebrutinib or pharmaceutically acceptable salt thereof is administered in the form of one or more tablets or capsules.

Embodiment 27. The method of any one of embodiments 1 to 26, wherein the fenebrutinib or pharmaceutically acceptable salt thereof is administered in the form of two tablets twice daily, each tablet comprising about 100 mg fenebrutinib or an equivalent amount of a pharmaceutically acceptable salt thereof.

Embodiment 28. The method of any one of embodiments 1 to 27, wherein the free form of fenebrutinib is administered.

Embodiment 29. A compound for use in a method of treating primary progressive multiple sclerosis (PPMS) in a subject in need thereof, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

Embodiment 30. The compound for use of embodiment 29, wherein the method further comprises evaluating disability progression in the subject, wherein disability progression is evaluated using the Expanded Disability Status Scale (EDSS), the 9-Hole Peg Test (9-HPT), or the Timed 25-Foot Walk Test (T25FWT), or any combinations thereof.

Embodiment 31. The compound for use of embodiment 29 or 30, wherein the method further comprises evaluating the onset of composite 12-week confirmed disability progression (cCDP12), wherein onset of the cCDP12 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.
and wherein the progression event is confirmed at least 12 weeks after the initial progression.

Embodiment 32. The compound for use of any one of embodiments 29 to 31, wherein time to a progression event is increased, wherein the progression event is:
an increase from baseline in EDSS score of at least 1.0 point in a subject with a baseline EDSS score of less than or equal to 5.5 points; or
an increase from baseline in EDSS score of at least 0.5 point in a subject with a baseline EDSS score of greater than 5.5 points.

Embodiment 33. The compound for use of any one of embodiments 29 to 32, wherein time to a progression event is increased, wherein the progression event is increase of at least 20% from baseline in time to complete the 9-HPT.

Embodiment 34. The compound for use of any one of embodiments 29 to 33, wherein time to a progression event is increased, wherein the progression event is an increase of at least 20% from baseline in T25FWT.

Embodiment 35. The compound for use of any one of embodiments 29 to 34, wherein time to onset of CDP12, cCDP12, CDP24, or cCDP24 is increased in comparison to a subject with PPMS who is not administered fenebrutinib or a pharmaceutically acceptable salt thereof.

Embodiment 36. The compound for use of embodiment 35, wherein the subject with PPMS who is not administered fenebrutinib is administered an anti-CD20 antibody.

Embodiment 37. The compound for use of any one of embodiments 32 to 36, wherein the time to a progression event or time to onset is increased at least 10%.

Embodiment 38. A compound for use in a method of slowing the progression of PPMS in a subject in need thereof, wherein the compound is fenebrutinib or a pharmaceutically acceptable salt thereof, and wherein the compound comprises administering to the subject about 200 mg fenebrutinib twice per day, or an equivalent amount of a pharmaceutically acceptable salt thereof.

Embodiment 39. The compound for use of embodiment 38, wherein the progression of PPMS is evaluated using the MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels.

Embodiment 40. The compound for use of embodiment 38 or 39, wherein the progression of PPMS comprises at least one progression event.

Embodiment 41. A compound for use in a method of delaying the onset of at least one progression event in a subject with PPMS, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

Embodiment 42. A compound for use in a method of reducing the risk of a subject with PPMS having at least one progression event, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

Embodiment 43. The compound for use of any one of embodiments 40 to 42, wherein the at least one progression event is selected from the group consisting of
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.

Embodiment 44. The compound for use of embodiment 43, wherein the progression event is confirmed at least 12 weeks after the initial progression.

Embodiment 45. The compound for use of any one of embodiments 38 to 40, 43, or 44, wherein the progression is slowed by at least 10% compared to another subject with PPMS who is not administered fenebrutinib or a pharmaceutically acceptable salt thereof and is administered an anti-CD20 antibody.

Embodiment 46. The compound for use of any one of embodiments 41, 43, or 44, wherein the onset of at least one progression event is delayed by at least 10% compared to another subject with PPMS who is not administered fenebrutinib or a pharmaceutically acceptable salt thereof and is administered an anti-CD20 antibody.

Embodiment 47. The compound for use of any one of embodiments 42 to 44, wherein the risk of having at least one progression event is decreased by at least 10% compared to another subject with PPMS who is not administered fenebrutinib or a pharmaceutically acceptable salt thereof and is administered an anti-CD20 antibody.

Embodiment 48. The compound for use of any one of embodiments 38 to 47, wherein the progression is slowed, or the onset is delayed, or the risk is decreased, in comparison to a subject with PPMS that is not administered fenebrutinib or a pharmaceutically acceptable salt thereof.

Embodiment 49. A compound for use in a method of reducing disability in a subject with PPMS, wherein the compound is fenebrutinib or a pharmaceutically acceptable salt thereof, and wherein the method comprises administering to the subject about 200 mg fenebrutinib twice per day, or an equivalent amount of a pharmaceutically acceptable salt thereof.

Embodiment 50. The compound for use of embodiment 49, wherein reducing disability comprises:
reducing the psychological impact of MS;
increasing upper limb function;
increasing walking ability;
decreasing fatigue;
improving work status; or
decreasing global impression of MS severity;
or any combinations thereof.

Embodiment 51. The compound for use of any one of embodiments 29 to 50, wherein the subject has a reduction in one or more symptoms of PPMS after beginning treatment with fenebrutinib, or a pharmaceutically acceptable salt thereof.

Embodiment 52. The compound for use of any one of embodiments 29 to 51, wherein the method further comprises the step of measuring one or more clinical or laboratory endpoints in the subject in order to evaluate the efficacy of treating PPMS.

Embodiment 53. The compound for use of embodiment 52, wherein the one or more clinical or laboratory endpoints are selected from the group consisting of the subject's MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels.

Embodiment 54. The compound for use of any one of embodiments 29 to 53, wherein the fenebrutinib or pharmaceutically acceptable salt thereof is administered orally.

Embodiment 55. The compound for use of any one of embodiments 29 to 54, wherein the fenebrutinib or pharmaceutically acceptable salt thereof is administered in the form of one or more tablets or capsules.

Embodiment 56. The compound for use of any one of embodiments 29 to 55, wherein the fenebrutinib or pharmaceutically acceptable salt thereof is administered in the form of two tablets twice daily, each tablet comprising about 100 mg fenebrutinib or an equivalent amount of a pharmaceutically acceptable salt thereof.

Embodiment 57. The compound for use of any one of embodiments 29 to 56, wherein the free form of fenebrutinib is administered.

Embodiment 58. A compound for use in the manufacture of a medicament for any of the methods of embodiments 1 to 28, wherein the compound is fenebrutinib or a pharmaceutically acceptable salt thereof.

### EXAMPLES

The present disclosure will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: A Phase III Multicenter, Randomized, Double-Blind, Double-Dummy, Parallel-Group Study to Evaluate the Efficacy and Safety of Fenebrutinib Compared with Ocrelizumab in Adult Patients with Primary Progressive Multiple Sclerosis

This Phase 111 study will examine the efficacy and safety of fenebrutinib compared with ocrelizumab in adult subjects with PPMS. Specific objectives and corresponding endpoints for the study are outlined below.

Study Objectives

### Primary, Efficacy Objective

The primary efficacy objective for this study is to evaluate the efficacy of fenebrutinib compared with ocrelizumab in patients with PPMS regardless of adherence to randomized treatment on the basis of the following endpoint:
Time to onset of cCDP12, which is the time from baseline to the first occurrence of a progression event according to at least one of the following three criteria, and must be confirmed at a regularly scheduled visit that is at least 12 weeks after the initial disability progression:
   An increase from baseline in EDSS score of ≥1.0 point in patients with a baseline EDSS score of ≤0.5 points or ≥0.5 point in patients with a baseline EDSS score of >5.5 points (confirmed disability progression [CDP])
   ≥20% increase from baseline in time to complete the 9-HPT
   ≥20% increase from baseline in T25FWT

### Secondary Efficacy Objective

The secondary efficacy objective for this study is to evaluate the effectiveness of fenebrutinib treatment compared with ocrelizumab on the basis of the following endpoints. The secondary endpoints do not reflect order of statistical hierarchy.
Time to onset of composite 24-week CDP (cCDP24)
Time to onset of 12-week CDP (CDP12), which is an increase from baseline EDSS score of ≥1.0 point in patients with a baseline EDSS score of ≤5.5 points or ≥0.5 point and in patients with a baseline EDSS score of >5.5 points
Time to onset of 24-week CDP (CDP24)
Percent change in total brain volume from Week 24 to Week 120 as assessed by MRI scan
Change from baseline in patient-reported physical impacts of MS (as measured by Multiple Sclerosis Impact Scale, 29-Item [MSIS-29] physical scale) at Week 120
Time to onset of 12-week confirmed 4-point worsening in Symbol Digit Modality Test (SDMT) score

### Exploratory Efficacy Objective

The exploratory efficacy objective for this study is to evaluate the efficacy of fenebrutinib compared with ocrelizumab and may include, but is not limited to, the following endpoints:
Proportion of patients with worsening in SDMT by 4 points
Change from baseline and proportion of patients with a meaningful deterioration from baseline at Week 120 for the following patient-reported outcomes (PROs):
   Psychological impacts of MS (MSIS-29 psychological scale)
   Upper limb function (Quality of Life in Neurological Disorders [Neuro-QoL-™], Upper Extremity Function Form)
   Walking (12-Item Multiple Sclerosis Walking Scale [MSWS-12])
   Fatigue (Patient-Reported Outcomes Measurement Information System-Fatigue Short Form for Multiple Sclerosis [PROMIS®-FatigueMS])
   Work status (Work Productivity and Activity Impairment [WPAI]:MS v2.0)
   Global impression of MS Severity (Patient Global Impression of Severity [PGI-S])
Time to onset of ≥20% increase in 12-week confirmed T25FWT
Time to onset of ≥20% increase in 12-week confirmed 9-HPT
Time to onset of ≥20% increase in 24-week confirmed T25FWT
Time to onset of ≥20% increase in 24-week confirmed 9-HPT
Proportion of patients with a meaningful deterioration from baseline in patient-reported physical impacts of MS (MSIS-29 physical scale) at Week 120
Proportion of patients with a meaningful deterioration from baseline in patient-reported psychological impacts of MS (MSIS-29 psychological scale) at Week 120
Total number of new T1Gd+ lesions from the baseline as detected by brain MRI scan^{a}
Total number of new/enlarging T2-weighted lesions as detected by MRI scan^{a}
Total number of new T1-hypointense lesions (black holes) from baseline as detected by MRI scan^{a}
Percent change from screening to Week 120 in serum neurofilament light chain (NfL) levels
Proportion of patients free of disability progression (cCDP12, cCDP24, CDP12, and CDP24) at Week 120 and at the time of clinical cutoff of primary analysis ^{a}In this study, the screening MRI measurements are used as the baseline measurements.

### Safety Objective

The safety objective for this study is to evaluate the safety of fenebrutinib compared with ocrelizumab on the basis of the following endpoints:
The nature, frequency, timing, and severity of adverse events: serious adverse events; and adverse events leading to study treatment withdrawal
Change from baseline in targeted vital signs
Change from baseline in targeted ECG parameters
Change from baseline in clinical laboratory results following study treatment administration
Change from baseline in the Columbia-Suicide Severity Rating Scale (C-SSRS)

### Pharmacokinetic Objectives

The pharmacokinetic (PK) objective for this study is to characterize the fenebrutinib PK profile on the basis of the following endpoint:
Plasma concentration of fenebrutinib at specified timepoints

Sparse PK samples will be collected in all patients. However, to better characterize fenebrutinib pharmacokinetics in patients with MS, more intensive PK samples will be collected in a small subset of patients.

The exploratory PK objectives for this study are as follows:
To evaluate potential relationships between drug exposure and the efficacy and safety of fenebrutinib on the basis of the following endpoints:
   Relationship between plasma concentrations of fenebrutinib and efficacy endpoints
   Relationship between plasma concentrations of fenebrutinib and safety endpoints
To evaluate potential relationships between selected covariates and exposure to fenebrutinib on the basis of the following endpoint:
   Relationship between selected covariates and plasma concentrations of fenebrutinib

### Biomarker Objectives

The exploratory biomarker objective for this study is to identify and/or evaluate biomarkers that are predictive of response to fenebrutinib (i.e., predictive biomarkers), are early surrogates of efficacy, are associated with progression to a more severe disease state (i.e., prognostic biomarkers), are associated with acquired resistance to fenebrutinib, are associated with susceptibility to developing adverse events or can lead to improved adverse event monitoring or investigation (i.e., safety biomarkers, including human leukocyte antigen [HLA] genotyping), can provide evidence of fenebrutinib activity (i.e., pharmacodynamic [PD] biomarkers), or can increase the knowledge and understanding of disease biology and drug safety, on the basis of the following endpoints:
Relationship between baseline biomarkers in blood (serum and/or plasma and/or RNA) and efficacy, PK, or other biomarker endpoints
Relationship between change from baseline to post-treatment sampling in blood biomarkers (serum and/or plasma and/or RNA) and efficacy, PK, or other biomarker endpoints
Relationship between genetics, including, but not limited to, HLA genotype, and efficacy, PK, or other biomarker endpoints

### Health Status Utility Objective

The exploratory health status utility objective for this study is to evaluate health status utility scores of patients treated with fenebrutinib on the basis of the following endpoint:
Relationship between EuroQol 5-Dimension, 5-Level Questionnaire (EQ-5D-5L) index score and clinical measurements that may support pharmacoeconomic modeling

### Detailed Study Design

This is a Phase 111, randomized, multicenter, double-blind, double-dummy, parallel-group study to evaluate the efficacy and safety of fenebrutinib on disability progression in adult patients with PPMS. All eligible patients will be randomized 1:1 to either daily oral fenebrutinib (or placebo) (200 mg, twice a day [BID]) or IV ocrelizumab (or placebo) (600 mg every 24 weeks) in a blinded fashion through an interactive voice or web-based response system. Approximately 946 patients will be enrolled and will be recruited globally. Patients who discontinue study medication early or discontinue from the study will not be replaced.

This study will consist of the following phases: screening (for up to 4 weeks), double-blind treatment (DBT) phase, DBT safety follow-up (DBT-SFU), optional open-label extension (OLE), and OLE safety follow-up (OLE-SFU) phase.

The study duration will vary for each patient as a result of the primary analysis being event driven.

### Study Design

This study will consist of the following:
Screening phase, for approximately 4 weeks
Double-blind treatment (DBT) phase, patients will be randomized to a 1:1 ratio of either 200 mg BID oral fenebrutinib or 600 mg IV ocrelizumab.
DBT safety follow-up (DBT-SFU), is available to patients who 1) remained on study treatment at the end of the DBT phase and do not wish to participate in the OLE or 2) have discontinued study treatment but have fewer than 24 weeks of follow up from their last ocrelizumab DBT infusion or fewer than 8 weeks of follow-up from their last dose of DBT fenebrutinib, whichever applies. Patients will be followed for safety for approximately 48 weeks.
Optional open-label extension (OLE) phase, if the primary analysis is positive, will be available for eligible patients. Patients will receive approximately 96 weeks of open-label fenebrutinib; however, the OLE phase may be extended.
OLE safety follow-up (OLE-SFU) phase, is available to patients who discontinue OLE fenebrutinib early or who complete the OLE phase. Patients will be followed in the OLE-SFU for approximately 8 weeks.

The study duration will vary for each patient as a result of the primary analysis being event driven. Patient safety will be monitored by an iDMC both for the initial safety assessment (iDMC-ISA) and at regular intervals throughout the DBT phase.

### Screening Phase

The screening phase will be approximately 4 weeks. Patients who fail the initial screening may qualify for one re-screening opportunity (for a total of two screenings per patient). Procedures at screening will include collection of medical history, physical examination, complete neurological examination, EDSS score, 9-HPT, T25FWT, ECG, MRI scan, and blood and urine samples.

### Double-Blind Treatment Phase

The duration of the DBT phase is partially event-driven. The primary analysis will occur when approximately 486 cCDP12 events have occurred and when all patients have participated in the DBT phase for at least 120 weeks. The DBT phase is considered completed when the results of the primary analysis are disclosed and the study becomes unblinded to sites. If the projected number of cCDP events (486) has not been reached when the last patient completes Week 120 in the DBT phase because of slower than anticipated disability progression rates, the DBT phase will be extended until the required number of cCDP12 events have occurred, to maintain statistical power to detect a treatment difference. The DBT phase may extend beyond 120 weeks for the initial group of patients enrolled in the study, based on the final length of the recruitment period for the study.

Patients who discontinue study treatment for any reason during the DBT phase will remain in the DBT phase but will not receive study treatment. These patients will continue to attend the DBT visits as scheduled but will have abbreviated efficacy and safety assessments.

Patients who discontinue study treatment during the DBT phase will be allowed to start another disease-modifying therapy (DMT), at the discretion of the patient and the investigator, after waiting at least 24 weeks from the last ocrelizumab/placebo DBT infusion or at least 8 weeks after the last DBT fenebrutinib/placebo administration, whichever is longer.

Unscheduled visits for the assessment of potential disease progression, new neurological symptoms, or safety events may occur at any time. Patients with new neurological symptoms suggestive of MS relapse or MS worsening should have an EDSS, 9-HPT, and T25FWT performed by the examining investigator within 7 days from the onset of the new neurological symptoms.

### Double-Blind Treatment Phase Safety Follow-Up

At the completion of the DBT phase, patients will enter a DBT-SFU for approximately 48 weeks if patients 1) remained on study treatment at the end of the DBT phase and do not wish to participate in the OLE or 2) have discontinued DBT study treatment but have had fewer than 24 weeks of follow-up since their last ocrelizumab DBT infusion or fewer than 8 weeks of follow-up since their last dose of DBT fenebrutinib, whichever applies. Patients will be followed for safety for approximately 48 weeks. Patients may begin on another DMT at the discretion of the investigator and patient after at least 24 weeks since the last ocrelizumab DBT infusion or at least 8 weeks since the last DBT fenebrutinib administration, as applicable.

### Optional Open-Label Extension

At the end of the DBT phase (disclosure of the DBT results and unblinding to sites), if the primary analysis and the benefit-risk assessment of the use of fenebrutinib therapy are positive, an optional OLE phase is planned for eligible patients who complete the DBT phase and, in the opinion of the investigator, could benefit from fenebrutinib treatment.

Additional unscheduled visits for the assessment of potential disease progression, new neurological symptoms, or safety events may occur at any time. Patients with new neurological symptoms suggestive of MS relapse or MS worsening should have an EDSS, 9-HPT, and T25FWT performed by the examining investigator (within 7 days from the onset of the new neurological symptoms).

### Open-Label Extension and Follow-Up

Patients who discontinue OLE fenebrutinib early or who complete the OLE phase will enter the OLE-SFU. Patients will be followed for safety for approximately 8 weeks. Only safety assessments will be collected during the OLE-SFU. Laboratory and safety assessments for the OLE-SFU phase will be performed at clinic visits approximately 4 weeks apart.

### Patient Population

Approximately 946 patients with PPMS will be enrolled in this study. Dynamic enrollment caps may be added to ensure that distribution of patients according to stratification factors will be balanced across region, screening EDSS score, and the presence or absence of T1Gd+ at screening.

### Inclusion Criteria

Patients must meet the following criteria for study entry:
Signed Informed Consent Form
Age 18-65 years inclusive at time of signing Informed Consent Form
Ability to comply with the study protocol
A diagnosis of PPMS in accordance to the revised 2017 McDonald Criteria (Tompson et al. 2018):
   Progressive disease from the onset. Superimposed relapses can occur in PPMS; therefore, clinical evidence from the investigator to the Sponsor is required to confirm the diagnosis of PPMS.
   One year of disability progression (retrospectively or prospectively determined) independent of clinical relapse provided as a summary of clinical evidence from the investigator to Sponsor is required to confirm a progressive stage for at least 12 months prior to randomization.
Plus two of the following criteria:
   Documented evidence of one or more T2-hyperintense lesions characteristic of MS in one or more of the following brain regions: periventricular, cortical or juxtacortical, or infratentorial (established by a historical MRI scan)
   Documented evidence of two or more T2-hyperintense lesions in the spinal cord (established by a historical MRI scan)
   Documented evidence of the presence of cerebrospinal fluid-specific oligoclonal bands (established by a historical lumbar puncture)
EDSS score from 3.0 to 6.5 inclusive at screening
For patients currently receiving proton pump inhibitors (PPIs) or H2-receptor antagonists (H2RAs): treatment at a stable dose during the screening period prior to the initiation of study treatment and plans to remain at a stable dose for the duration of study treatment
Patients must not initiate PPIs or H2RAs within 2 weeks of randomization.
For patients requiring symptomatic treatment for MS (e.g., fampridine, cannabis) and/or physiotherapy: treatment at a stable dose/regimen during the screening period prior to the initiation of study drug and plans to remain at a stable dose/regimen for the duration of study treatment
Patients must not initiate symptomatic treatment for MS or physiotherapy within 4 weeks of randomization.
Neurologically stable for at least 30 days prior to randomization and baseline assessments
Ability to complete the 9-HPT for each hand in <240 seconds
Ability to perform T25FWT
For women of childbearing potential: agreement to remain abstinent (refrain from heterosexual intercourse) or use contraceptive methods with a failure rate of <1% per year during the treatment period and for 6 or 12 months (as applicable by the local label for ocrelizumab) after the final dose of study medication. Women must refrain from donating eggs during this same period. Hormonal contraceptive methods must be supplemented by a barrier method.
For men: agreement to remain abstinent (refrain from heterosexual intercourse) or use a condom, and agreement to refrain from donating sperm

### Exclusion Criteria

Patients who meet any of the following criteria will be excluded from study entry:
Any known or suspected active infection at screening or baseline, or any major episode of infection requiring hospitalization or treatment with IV anti-microbials within 8 weeks prior to and during screening or treatment with oral anti-microbials within 2 weeks prior to and during screening
History of confirmed or suspected progressive multifocal leukoencephalopathy
History of cancer, including hematologic malignancy and solid tumors, within 10 years of screening. Basal or squamous cell carcinoma of the skin that has been excised and is considered cured and in situ carcinoma of the cervix treated with apparent success by curative therapy>1 year prior to screening is not exclusionary.
Immunocompromised state, defined as one or more of the following:
   CD4 count<250/μL or ANC<1.5×103/μL or serum IgG<4.6 g/L
Known presence of other neurological disorders, including, but not limited to, the following:
   History of ischemic cerebrovascular disorders (e.g., stroke, transient ischemic attack, spontaneous intracranial hemorrhage, or traumatic intracranial hemorrhage) or ischemia of the spinal cord
   History or known presence of CNS or spinal cord tumor (e.g., meningioma, glioma)
   History or known presence of potential metabolic causes of myelopathy (e.g., untreated vitamin B12 deficiency)
   History or known presence of infectious causes of myelopathy (e.g., syphilis, Lyme disease, HTLV-1, herpes zoster myelopathy)
   History of genetically inherited progressive CNS degenerative disorder (e.g., hereditary paraparesis, mitochondrial myopathy, encephalopathy, lactic acidosis, stroke syndrome)
   Neuromyelitis optica spectrum disorder
   History or known presence of systemic autoimmune disorders potentially causing progressive neurological disease (e.g., lupus, anti-phospholipid antibody syndrome, Sjögren syndrome, Behçet disease)
   History or known presence of sarcoidosis
   History of severe, clinically significant brain or spinal cord trauma (e.g., cerebral contusion, spinal cord compression)
Evidence of clinically significant cardiovascular (including arrhythmias or QTc prolongation), psychiatric, pulmonary, renal, hepatic, endocrine (including uncontrolled diabetes, non-gallstone pancreatitis, or chronic pancreatitis), metabolic, or gastrointestinal (GI) disease that, in the investigator's opinion, would preclude patient participation
Patients meeting the New York Heart Association Class III and Class IV criteria for congestive heart failure
Screening 12-lead ECG that demonstrates clinically relevant abnormalities that may affect patient safety or interpretation of study results including QT interval corrected through use of Fridericia's formula>440 ms demonstrated by at least two ECGs>30 minutes apart
Current treatment with medications that are well known to prolong the QT interval at doses that have a clinically meaningful effect on QT, as determined by the investigator
History of ventricular dysrhythmias or risk factors for ventricular dysrhythmias such as long QT syndrome and other genetic risk factors (e.g., Brugada syndrome); structural heart disease; coronary heart disease (symptomatic or with ischemia demonstrated by diagnostic testing, prior coronary artery bypass grafting, or coronary lesions>70% diameter stenosis that have not been or cannot be re-vascularized); clinically significant electrolyte abnormalities (e.g., hypokalemia, hypomagnesemia, hypocalcemia); family history of sudden, unexplained death; or cardiac ion channel genetic mutations (e.g., congenital long QT syndrome)
Any concomitant disease that may require chronic treatment with systemic corticosteroids or immunosuppressants during the course of the study
History of alcohol or other drug abuse within 12 months prior to screening
Pregnant or breastfeeding, or intending to become pregnant during the study or 6 or 12 months (as applicable from the local label for ocrelizumab) after final dose of study drug
   All women of childbearing potential will have a serum pregnancy test at screening. Urine pregnancy tests will be performed locally at specified subsequent visits. If a urine pregnancy test is positive, it must be confirmed by a serum pregnancy test (performed locally).
Positive screening tests for active, latent, or inadequately treated hepatitis B (as evidenced by either of the following):
   Positive hepatitis B surface antigen
   Positive hepatitis B core antibody [total HBcAb] and detectable Hep B virus DNA
Positive screening tests for hepatitis C (positive hepatitis C antibodies).
Evidence of active or latent or inadequately treated infection with tuberculosis (TB) as defined by the following:
   A positive QuantiFERON TB-Gold® (QFT) test at screening or within the 3 months prior to screening is required. If QFT is unavailable, a negative Mantoux purified protein derivative skin test, as defined by the Centers for Disease Control and Prevention guidelines, may be performed at the screening visit or within the 3 months prior to screening and read locally.
   Patients with a history of Bacille Calmette-Guerin vaccination should be screened using the QFT test only.
   An indeterminate QFT test should be repeated.
   A positive QFT test or two successive indeterminate QFT results should be considered positive diagnostic TB test.
   An indeterminate QFT test followed by a negative QFT test should be considered a negative diagnostic TB test.
Abnormalities in hepatic synthetic function tests (e.g., PT, INR, PTT, albumin) judged by the investigator to be clinically significant
History of hospitalizations or transfusion for a GI bleed
Known bleeding diathesis
Any condition possibly affecting oral drug absorption
History of or currently active primary or secondary (non-drug-related) immunodeficiency, including known history of HIV infection or IgG<500 mg/dL
Contraindications to mandatory premedications (i.e., corticosteroids and antihistamines) for infusion-related reactions (IRRs), including:
   Uncontrolled psychosis for corticosteroids
   Closed-angle glaucoma for antihistamines
Inability to complete an MRI scan (contraindications for MRI scan, including but not restricted to, pacemaker, cochlear implants, intracranial vascular clips, surgery within 6 weeks of entry in the study, coronary stent implanted within 8 weeks prior to the time of the intended MRI scan) or contraindication to gadolinium administration
Lack of peripheral venous access
Any previous treatment with bone marrow transplantation and hematopoietic stem cell transplantation
Any previous history of transplantation or anti-rejection therapy
Systemic corticosteroid therapy within 4 weeks prior to screening
   The screening period may be extended for patients who have used systemic corticosteroids for MS before screening. For a patient to be eligible, systemic corticosteroids must not be administered between screening and baseline.
Treatment with IV Ig or plasmapheresis within 12 weeks prior to randomization
Sensitivity or intolerance to any ingredient (including excipients) of fenebrutinib or ocrelizumab
Receipt of a live or live-attenuated vaccine within 6 weeks prior to randomization. Influenza vaccination is permitted if the inactivated vaccine formulation is administered.
Need for systemic anti-coagulation (oral or injectable) or anti-platelet agent other than nonsteroidal anti-inflammatory drugs, aspirin, and other salicylates (aspirin up to 162 mg once daily is allowed)
Previous treatment with fenebrutinib or another Bruton's tyrosine kinase inhibitor for any indication
Treatment with any investigational agent (including high-dose biotin) within 24 weeks prior to screening (Visit 1) or 5 half-lives of the investigational drug (whichever is longer), or treatment with any experimental procedure for MS (e.g., treatment for chronic cerebrospinal venous insufficiency)
Requirement for any prohibited concomitant medications
Use of previous B-cell targeting therapies (including alemtuzumab). Patients with historical treatment with an anti-CD20 who have CD19+ B cell levels within normal range at screening and at baseline as determined by screening and baseline results from the study central laboratory may be eligible.
Previous use of fingolimod or another sphingosine-1-phosphate receptor modulators within 3 months of randomization^{a}
Previous use of cladribine within 12 months of randomization^{a}
Previous use of natalizumab for more than 1 year and within 6 months of randomization^{a}
Previous treatment with mycophenolate mofetil, methotrexate, mitoxantrone, dimethyl fumarate, glatiramer acetate, interferons, teriflunomide, or laquinimod within 3 months of randomization^{a}
   Patients previously treated with teriflunomide will require the appropriate elimination protocol (as per local label).
Any previous treatment with daclizumab, alemtuzumab, or cyclophosphamide
Previous treatment with any other immunomodulatory or immunosuppressive medication not already listed above without appropriate washout as described in the applicable local label. If the washout requirements are not described in the applicable local label, then the wash out period must be 5 times the half-life of the medication. The PD effects of the previous medication must also be considered when determining the required time for washout.^{a}
Having one or more of the following laboratory results:
   Estimated glomerular filtration rate (eGFR)<60 mL/min/1.73 m² (may be repeated if eGFR 45-59 mL/min/1.73 m²)
   ALT or AST>2×ULN (may be repeated if 2-3×ULN)
   Total bilirubin greater than 1.5×ULN (may be repeated if 1.6-3×ULN), with the exception for patients with Gilbert's disease
   Hemoglobin<9.5 g/dL (may be repeated if 9-9.4 g/dL)
   Platelet count<100×10⁹/L (may be repeated if 80-100×10⁹/L)
   Abnormalities in hepatic synthetic function tests (e.g., PT, INR, PTT, albumin) judged by the investigator to be clinically significant ^{a}Patients screened for this study should not be withdrawn from therapies for the sole purpose of meeting eligibility for the trial. Patients who discontinue their current therapy for non-medical reasons should specifically be informed of their treatment options before deciding to enter the study.

### Eligibility Criteria for Open-Label Extension Phase

Patients who meet the following criteria may participate in the OLE phase:
Completed the DBT phase of the study (remaining on study treatment; no other DMT administered) and who, in the opinion of the investigator, may benefit from treatment with fenebrutinib
Able and willing to provide written informed consent to participate in the OLE phase and to comply with the study protocol
For women of childbearing potential: agreement to remain abstinent (refrain from heterosexual intercourse) or use contraceptive methods with a failure rate of <1% per year during the treatment period and for at least 28 days after the final dose of fenebrutinib. Women must refrain from donating eggs during this same period.
For men: agreement to remain abstinent (refrain from heterosexual intercourse) or use a condom, and agreement to refrain from donating sperm.

### End of Study

The end of this study is defined as the date when the last patient, last visit (LPLV) occurs in the OLE phase or the LPLV in the OLE-SFU phase, whichever occurs later.

### Length of Study

The duration of the DBT phase will be approximately 225 weeks or approximately 4.7 years (assuming the last patient is randomized after 122 weeks of recruitment+120 weeks of DBT phase for the last patient enrolled in the study). The maximum length of the study, from screening of the first patient to the end of the study, is expected to be approximately 370 weeks or approximately 7 years (assuming 122 weeks of recruitment+120 weeks of DBT phase+24 weeks washout+96 weeks of OLE fenebrutinib treatment+8 weeks of OLE-SFU for the last patient enrolled in the study).

In addition, the Sponsor may decide to terminate the study at any time or extend the duration of the OLE.

### Investigational Medicinal Products

### Fenebrutinib or Placebo

Patients will take two 100 mg tablets orally BID for a total dose of 400 mg of fenebrutinib (or placebo) every day. Patients will self-administer two 100 mg tablets in the morning and two 100 mg tablets in the evening by mouth. Fenebrutinib (or placebo) may be taken orally with or without food. Administration of study drug should be staggered with antacid use (i.e., study drug should be taken 2 hours before or 2 hours after antacid administration). Patients should be instructed that a missed dose should not be taken with the next scheduled dose.

For patients participating in the intensive PK sampling subgroup, the fenebrutinib (or placebo) morning dose on Days 1 and 15 will be administered at the morning (mandatory) clinical visit while the patient is fasting. Patients should be instructed that a missed dose should not be taken with the next scheduled dose.

### Ocrelizumab or Placebo

Patients will be administered IV infusions of 600 mg ocrelizumab (or placebo) every 24 weeks. The first dose of ocrelizumab (or placebo) will be administered as two 300-mg IV infusions given 14 days apart. For the subsequent doses, ocrelizumab (or placebo) will be administered as a single 600-mg IV infusion every 24 weeks. A minimum interval of 22 weeks must be maintained between each single infusion. Each ocrelizumab (or placebo) 300-mg dose should be administered as a slow IV infusion over approximately 2.5 hours. Each ocrelizumab 600-mg dose should be administered as a slow IV infusion over approximately 3.5 hours.

All patients must receive mandatory prophylactic treatment with 100 mg of methylprednisolone administered by slow IV infusion, to be completed approximately 30 minutes before the start of each ocrelizumab (or placebo) infusion. Use of an equivalent dose of an alternative steroid should be used as premedication prior to the infusion in the case when the use of methylprednisolone is contraindicated for the patient. Additionally, a mandatory oral or IV antihistaminic drug (such as IV diphenhydramine 50 mg or an equivalent dose of an alternative) must be administered approximately 30-60 minutes prior to the start of each ocrelizumab (or placebo) infusion. An analgesic/antipyretic such as acetaminophen/paracetamol (1 g) can also be considered.

### Statistical Methods

Primary Analysis

The primary efficacy endpoint for this trial is time to onset of cCDP12, defined as the time from baseline to first cCDP12. The independent examining investigator at each study site will assess the components of the cCDP (EDSS, 9-HPT, and T25FWT) for all patients at the site at screening, baseline, regularly scheduled visits during the DBT phase, at unscheduled visits, and at the Treatment Discontinuation visit. The independent examining investigator is not the physician responsible for the patient care (i.e., the treating investigator).

The time to onset of cCDP12 in the fenebrutinib arm and ocrelizumab arm will be compared and tested using a stratified log-rank test with the null and alternative hypotheses as follows:
H₀: There is no difference in the time to onset of cCDP12 between the fenebrutinib and ocrelizumab groups.
H₁: There is a difference in the time to onset of cCDP12 between the fenebrutinib and ocrelizumab groups.

The proportion of patients with cCDP12 over time will be estimated using Kaplan-Meier methodology and the overall hazard ratio will be estimated using a stratified Cox proportional hazards model.

The specific composite component that generated the initial composite disability progression event is required for confirmation of cCDP. All assessments between initial event and the confirmation visit need to satisfy the definition of a composite disability progression event to be confirmed. Assessments occurring within 30 days after a protocol-defined relapse will not be used for confirmation of initial disease progression. Patients who prematurely discontinue study drug will be asked to continue with the study-specified assessments, and every effort will be made to follow up on their primary and secondary assessments at the next scheduled visit. All initial disability progression events with corresponding confirmation visits at the next scheduled visit w % ill be considered for the statistical analysis regardless of whether the patient discontinued study drug or the confirmation visit occurred during the DBT phase.

Determination of Sample Size

The purpose of this study is estimation and hypothesis testing regarding the effect of fenebrutinib on the time from baseline to cCDP12 relative to ocrelizumab. P-values and point and interval estimates of the true underlying hazard ratio will be obtained.

The sample size of this trial is based on testing the null hypothesis of no difference between the control and experimental arms. This study will enroll approximately 946 patients with an expected recruitment of over 122 weeks. The sample size of this study is driven by the primary efficacy analysis of cCDP12 and the following assumptions:
A two-group test of equal times to cCDP12
Two-sided type I error=0.05
Time to cCDP12 following an exponential distribution in each arm
50% of the patients in the control arm have cCDP12 events occurring by Week 120
A final analysis is based on approximately 486 cCDP12 events
Approximately 89% power when the true risk reduction=25% (i.e., hazard ratio=0.75)
20% dropout per arm by Week 120

### Example 2: A Phase III Multicenter, Randomized, Double-Blind, Double-Dummy, Parallel-Group Study to Evaluate the Efficacy and Safety of Fenebrutinib Compared with Ocrelizumab in Adult Patients with Primary Progressive Multiple Sclerosis

This Phase III study will examine the efficacy and safety of fenebrutinib compared with ocrelizumab in adult subjects with PPMS. The specific objectives, corresponding endpoints, inclusion criteria, exclusion criteria, and other aspects of this study are as described in Example 1 above, with the following additions and/or changes.
Primary Efficacy Objective: In assessing the time to onset of cCDP12: All assessments between initial event and the confirmation visit need to satisfy the definition of a composite disability progression event to be confirmed. Assessments occurring within 90 days after a protocol-defined relapse will not be used for confirmation of initial disease progression.
Secondary Efficacy Objectives:
   Percent change in total brain volume from Week 24 as assessed by MRI scan
   Change from baseline in patient-reported physical impacts of MS (as measured by Multiple Sclerosis Impact Scale, 29-Item IMSIS-291 physical scale)
An Exploratory Endpoint is added—change from baseline and proportion of patients with a meaningful deterioration from baseline at Week 120—Global impression of MS Change (Patient Global Impression of Change)
The Safety Objective, Change from baseline in the Columbia-Suicide Severity Rating Scale is changed to: Proportion of patients with suicidal ideation or behavior, as assessed by Columbia-Suicide Severity Rating Scale
Randomization will be stratified according to the following criteria:
   Global Region (United States vs. non-United States)
   EDSS score (5.0 vs. >5.0)
   MRI T1Gd+ at screening (presence or absence)
Inclusion Criteria:
   patients must be able and willing to provide a signed Informed Consent Form to participate.
   The Pre-Baseline Disability Progression Questionnaire will be used to confirm one year of disability progression independent of clinical relapse
Exclusion criteria:
   Patients with a previous history of a serious IRR (Common Terminology Criteria for Adverse Events Grade≥4) and/or any hypersensitivity reaction to ocrelizumab
   Gilbert's Syndrome is included in clinically significant hepatic disease that may, in the investigator's opinion, preclude patient participation
   Men intending to father a child during the study or 6 or 12 months (as applicable from the local label for ocrelizumab) after final dose of study drug
Exclusion criteria regarding concomitant and/or previously administered medications is updated to include:
   Treatment with strong CYP3A4 inhibitors, strong or moderate CYP3A4 inducers, within 7 days or 5 drug elimination half-lives (whichever is longer) prior to randomization
   Treatment with CYP3A4 substrates with a narrow therapeutic window within 7 days or 5 drug elimination half-lives (whichever is longer) prior to randomization
   Previous use of anti-CD20s, including ocrelizumab, within 6 months of randomization, and treatment discontinuation was not motivated by safety reasons or lack of efficacy
   Previous use of fingolimod, siponimod, or ozanimod within 8 weeks of randomization
   Previous use of natalizumab for more than 1 year and within 6 months of randomization
   Previous treatment with dimethyl fumarate, interferons, and glatiramer acetate within 4 weeks of randomization
   Previous treatment with mycophenolate mofetil or methotrexate12 weeks of randomization
   Previous treatment of teriflunomide, unless >/=24 months from screening or teriflunomide plasma concentrations are <0.02 mg/L at screening
   Any previous treatment with cladribine, mitoxantrone, daclizumab, alemtuzumab, or cyclophosphamide
Pregnancy Tests: Prior to Week 12, pregnancy tests will be performed at each scheduled clinic visit. After Week 12, women of childbearing potential should perform monthly urine pregnancy tests at home in addition to the urine pregnancy tests performed at each scheduled clinic visit. Urine pregnancy test kits will be provided to female patients at each scheduled clinic visit. If a patient becomes pregnant during the study, the patient must be instructed to immediately stop the study drug, inform the investigator, and come in for an unscheduled visit within 5 days of discovering the pregnancy. A positive at-home urine pregnancy test result must be confirmed by serum pregnancy test, preferably from the central laboratory. At-home pregnancy tests are not required beyond 28 days after permanently discontinuing study treatment.
If pregnancy is detected, it must be confirmed by a serum pregnancy test. If pregnancy is confirmed, the patient must permanently discontinue study drug.
In the Double-blind treatment (DBT) phase, patients will be randomized to a 1:1 ratio of either 200 mg BID oral fenebrutinib (or placebo) or 600 mg IV ocrelizumab (or placebo).
Semi-structured telephone interviews will be conducted during the DBT phase every 6 weeks (±3 days) between study visits from Week 24 onwards.
Semi-structured telephone interviews will be conducted in the OLE phase every 6 weeks (±3 days) between study visits from Week 24 onwards.

Prohibited Therapies

Medications in the following categories should be prohibited for 7 days or 5 half-lives, whichever is longer, prior to the first dose of study drug until the final dose of study drug:
Strong CYP3A4 inhibitors
Strong or moderate CYP3A inducers

The following medications should be prohibited during study treatment:
CYP3A4 substrates with a narrow therapeutic window
   Table 1 summarizes a list of prohibited medications. This list is not comprehensive:

TABLE 1
summarizes a list of prohibited medications. This list is not comprehensive:

| Class | Examples of Drugs in this Class |
|---|---|
| Strong CYP3A4 inhibitors | Boceprevir, cobicistat, clarithromycin, danoprevir/ritonavir, |
| | elvitegravir/ritonavir, indinavir/ritonavir, itraconazole, |
| | idelalisib, ketoconazole, lopinavir/ritonavir, nefazodone, |
| | nelfinavir, posaconazole, ritonavir, saquinavir, telaprevir, |
| | telithromycin, and voriconazole |
| Strong CYP3A inducers | Apalutamide, carbamazepine, enzalutamide, mitotane, |
| | phenytoin, rifampin, and hyperforin (St. John's Wort) |
| Moderate CYP3A inducers | Bosentan, dexamethasone, efavirenz, etravirine, |
| | phenobarbital, primidone, phenobarbital, and rifabutin |
| CYP3A4 substrate with a | Alfentanil, astemizole, cyclosporine, cisapride, |
| narrow therapeutic window | dihydroergotamine, ergotamine, everolimus, fentanyl, |
| | pimozide, quinidine, sirolimus, terfenadine, and tacrolimus |

Other Prohibited Therapies

Use of the following concomitant therapies is prohibited as described below for patients in the DBT phase who remain on study treatment, in the OLE phase, and in the OLE-SFU phase:
Investigational therapy (other than protocol-mandated study treatment)
Any B-cell targeted therapy (e.g., rituximab, alemtuzumab, atacicept, belimumab, ofatumumab, or commercial ocrelizumab)
BTK inhibitors (other than fenebrutinib)
Any other DMT for MS (including, but not limited to, high-dose biotin, cladribine, mitoxantrone, interferons, dimethyl fumarate and other fumarates, and fingolimod and other sphingosine-1-phosphate receptor modulators)
Systemic anti-coagulation (oral or injectable) or anti-platelet agent other than nonsteroidal anti-inflammatory drugs, aspirin, and other salicylates (aspirin up to 162 mg once daily is allowed)
Use of stand-alone doses of acid-reducing agents (e.g., PPIs, H2RAs) at visits requiring PK sampling is prohibited.

Use of the following concomitant therapy is prohibited as described below during the DBT phase for patients who discontinue DBT treatment and the DBT-SFU:
Investigational therapy (other than protocol-mandated study treatment)

Caution is advised when administering a DMT after fenebrutinib use. There are insufficient data available regarding the risk associated with switching from fenebrutinib to other products.

Table 2 summarizes a list of medications that may be administered concomitantly but such administration may include certain cautions. This list is not comprehensive:

TABLE 2
summarizes a list of medications that may be administered concomitantly but such
administration may include certain cautions. This list is not comprehensive:

| Class | Recommendation | Examples of Drugs in this Class |
|---|---|---|
| Antacids | Take fenebrutinib 2 hours before | Bismuth subsalicylate, calcium carbonate, |
| | or 2 hours after antacid | aluminum-magnesium hydroxide (e.g., Maalox ®, |
| | | Pepto-Bismol ®, Rolaids ®) |
| Breast | Use with caution and monitor for | Anti-hypertensive (prazosin) |
| cancer | adverse events related to BCRP | Anti-inflammatory (sulfasalazine) |
| resistance | substrates as directed by product | Lipid-lowering (rosuvastatin [recommended |
| protein | labeling | maximum dose: 10 mg/day], atorvastatin |
| (BCRP) | | [recommended maximum dose: 20 mg/day]) |
| substrates | | Muscle relaxants (dantrolene) |
| | | Steroids (estrone-3-sulfate) |
| Sensitive | Use with caution and monitor for | Antiemetic/prokinetic (aprepitant) |
| CYP3A | adverse events related to CYP3A | Anti-histamine (astemizole) |
| substrates | substrates as directed by product | Anti-hypertensive/cardiac (dronedarone, |
| | labeling | eplerenone, felodipine, nisoldipine, ticagrelor, |
| | | vardenafil) |
| | | Benzodiazepines (alprazolam, diazepam, |
| | | midazolam) |
| | | Lipid-lowering (simvastatin [recommended |
| | | maximum dose: 1 Omg/day], lovastatin |
| | | [recommended maximum dose: 20mg/day]) |
| | | Migraine (eletriptan, ergotamine) |
| | | Steroids (budesonide, fluticasone) |
| | | Other (buspirone, conivaptan, darifenacin, |
| | | dasatinib, lurasidone, quetiapine, sildenafil, |
| | | tolvaptan, triazolam) |

Assessment of Relapse

For this study, a relapse is the occurrence of new or worsening neurological symptoms attributed to MS and immediately preceded by a relatively stable of improving neurological state of at least 90 days. Symptoms must persist for >24 hours and should not be attributable to confounding clinical factors (e.g., fever, infection, injury, adverse reactions to concomitant medications). The new or worsening neurological symptoms must be accompanied by objective neurological worsening consistent with an increase of at least one of the following:
Half a step (0.5 point) on the EDSS
Two points on one of the selected FSS listed below
One point on two or more of the selected FSS listed below

The change must affect the following selected FSS: pyramidal, ambulation, cerebellar, brainstem, sensory, or visual. Episodic spasms, sexual dysfunction, fatigue, mood change, or bladder or bowel urgency or incontinence will not suffice to establish a relapse. Note that the following items need not be scored: sexual dysfunction and fatigue.

### Example 3: Comparison of In Vitro Properties of BTK Inhibitors

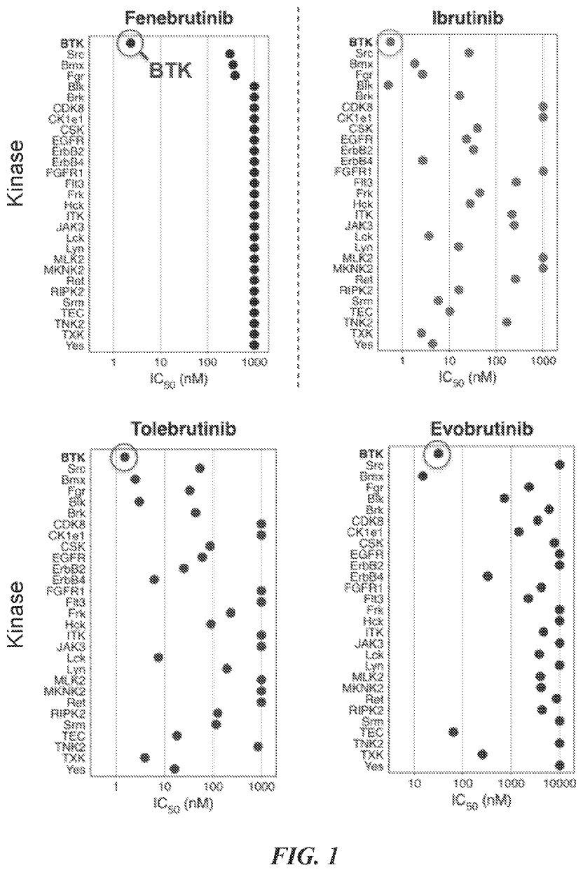

The in vitro properties of the three BTK inhibitors fenebrutinib, evobrutinib, and tolebrutinib are compiled in Table 3. Evobrutinib and tolebrutinib are covalent inhibitors, whereas fenebrutinib is a non-covalent inhibitor. The kinase selectivities of fenebrutinib, evobrutinib, tolebrutinib, and the covalent BTK inhibitor ibrutinib are also shown in FIG. 1.

BTK inhibitory potency (IC₅₀) and kinase selectivity of fenebrutinib (FEN), evobrutinib (EVO), and tolebrutinib (TOL) were assessed internally or in a commercial panel of over 200 human kinases. FEN, TOL, and EVO were screened at 1 μM, and EVO was also screened at 10 μM because it has a weaker BTK IC₅₀ than FEN and TOL. IC₅₀ values were determined for all kinases inhibited by at least 50% in the initial screen at 1 or 10 μM. To demonstrate that the selectivity values determined using the ICs, values are relevant for the covalent inhibitors EVO and TOL, their covalent reactivity, or k_{inact}/Kᵢ inactivation efficiency, was measured in biochemical assays by monitoring in real time the competition by the covalent inhibitors with a fluorescent active site ligand against BTK and BMX. FEN was also tested in human whole blood for its ability to block activation of B cells (CD69) and basophils (CD63). The rate of FEN release from the BTK•FEN complex was quantified in a biochemical preincubation-dilution experiment, where BTK activity was recovered with a rate constant kw and residence time 1/k_{off}.

FEN potently inhibits BTK (IC₅₀=2.3 nM); TOL inhibits BTK with IC₅₀=1.5 nM, whereas EVO is much less potent (IC₅₀=32 nM). In whole blood, FEN potently blocks activation of B cells (CD69 IC₅₀=8 nM) and basophils (CD63 IC₅₀=31 nM). In the kinase panel, FEN (1 μM) inhibits by >50% only 3/286 off-target kinases, whereas TOL (1 μM) inhibits 19/218 off-target kinases. EVO inhibits 3/221 off-target kinases at 1 μM, but at 10 μM it inhibits 18/218 kinases. Based on kinase IC₅₀ values. FEN is >130-fold selective against all 286 kinases tested, whereas EVO is <75-fold selective vs. Bmx (0.5×), TEC (2×), ErbB4 (10×), Blk (23×), and Flt3 (71×). TOL is <10-fold selective vs. BMX, BLK, ERBB4, TXK and LCK, and inhibits eleven additional kinases with <100-fold selectivity (Src, Fgr, TEC, RIPK2, BRK, CSK, YES, ERBB2, EGFR, FHCK, and SRM). The difference in kinase selectivity among the tested compounds is further illustrated in FIG. 1. In addition, the covalent kinetic selectivity of EVO and TOL as assessed by the ratio of k_{inact}/Kᵢ for BMX vs. BTK (EVO=0.5, TOL=1) was found to be nearly equal to the IC₅₀ selectivity for these inhibitors against these kinases (EVO=0.5, TOL=2). Finally, in a preincubation-dilution assay the BTK•FEN complex demonstrated high stability; FEN dissociates slowly from BTK and shows a residence time of 18.3 hours bound to BTK.

Table 3 summarizes in vitro properties of fenebrutinib, evobrutinib, and tolebrutinib

TABLE 3
summarizes in vitro properties of fenebrutinib, evobrutinib, and tolebrutinib

| | Parameter | Fenebrutinib | Evobrutinib | Tolebrutinib |
|---|---|---|---|---|
| Kinase | # off-target kinases >50% INH/# total | 2/286 ^{a} | 3/221 ^{a} | 19/218 ^{d} |
| Selectivity | kinases tested @ 1 μM | | | |
| | BTK IC₅₀, nM (fold selectivity) | 2.3 (1) ^{a} | 31.7 (1) ^{a} | 1.5 (1) ^{d} |
| | Src IC₅₀, nM (fold selectivity) | 302 (131) ^{a} | — | 54 (36) ^{d} |
| | BMX IC₅₀, nM (fold selectivity) | 351 (153) ^{a} | 15 (0.5) ^{d} | 2.5 (2) ^{d} |
| | Fgr IC₅₀, nM (fold selectivity) | 387 (168) ^{a} | 2,330 (74) ^{d} | 33 (22) ^{d} |
| | BLK IC₅₀, nM (fold selectivity) | — | 727 (23) ^{d} | 3.0 (2) ^{d} |
| | ERBB4 IC₅₀, nM (fold selectivity) | — | 326 (10) ^{d} | 6.1 (4) ^{d} |
| | FLT3 IC₅₀, nM (fold selectivity) | — | 2,250 (71) ^{d} | — |
| | TEC IC₅₀, nM (fold selectivity) | — | 64.1 (2) ^{d} | 18 (12) ^{d} |
| | TXK IC₅₀, nM (fold selectivity) | — | 254 (8) ^{d} | 3.9 (3) ^{d} |
| | CK1e1 IC₅₀, nM (fold selectivity) | — | 1,450 (46) ^{d} | — |
| | CDK8/cycC IC₅₀, nM (fold selectivity) | — | 3,500 (110) ^{d} | — |
| | LCK IC₅₀, nM (fold selectivity) | — | 3,800 (120) ^{d} | 7.5 (5) ^{d} |
| | MLK2 IC₅₀, nM (fold selectivity) | — | 3,980 (126) ^{d} | — |
| | MKNK2 IC₅₀, nM (fold selectivity) | — | 4,130 (130) ^{d} | — |
| | FGFR1 IC₅₀, nM (fold selectivity) | — | 4,150 (131) ^{d} | — |
| | RIPK2 IC₅₀, nM (fold selectivity) | — | 4,330 (137) ^{d} | 125 (83) ^{d} |
| | ITK IC₅₀, nM (fold selectivity) | — | 4,640 (146) ^{d} | — |
| | BRK IC₅₀, nM (fold selectivity) | — | 6,020 (190) ^{d} | 44 (29) ^{d} |
| | CSK IC₅₀, nM (fold selectivity) | — | 7,820 (247) ^{d} | 87 (58) ^{d} |
| | RET IC₅₀, nM (fold selectivity) | — | 8,630 (272) ^{d} | — |
| | YES IC₅₀, nM (fold selectivity) | — | — | 16 (11) ^{d} |
| | ERBB2 IC₅₀, nM (fold selectivity) | — | — | 25 (17) ^{d} |
| | EGFR IC₅₀, nM (fold selectivity) | — | — | 60 (40) ^{d} |
| | HCK IC₅₀, nM (fold selectivity) | — | — | 91 (61) ^{d} |
| | SRM IC₅₀, nM (fold selectivity) | — | — | 116 (77) ^{d} |
| | LYN IC₅₀, nM (fold selectivity) | — | — | 194 (129) ^{d} |
| | FRK IC₅₀, nM (fold selectivity) | — | — | 231 (154) ^{d} |
| | TNK2 IC₅₀, nM (fold selectivity) | — | — | 847 (565) ^{d} |
| Covalent | BTK Ki, nM | NA | 290 ^{d} | 8.7 ^{d} |
| Reaction | BTK k_{inact}, s⁻¹ | NA | 0.0052 ^{d} | 0.00063 ^{d} |
| | BTK k_{inact}/Kᵢ, M⁻¹ s⁻¹ | NA | 18,000 ^{d} | 58,100 ^{d} |
| Covalent | BTK Ki, nM | NA | 84 ^{d} | 42 ^{d} |
| Reaction | BTK k_{inact}, s⁻¹ | NA | 0.0032 ^{d} | 0.0029 ^{d} |
| | BTK k_{inact}/Kᵢ, M⁻¹ s⁻¹ | NA | 38,100 ^{d} | 60,800 ^{d} |
| Covalent | (BTK k_{inact}/Kᵢ)/(BMX k_{inact}/Kᵢ) | NA | 0.5 ^{d} | 1.0 ^{d} |
| Kinetic | | | | |
| Selectivity | | | | |
| Kinetics and | BTK residence time, h | 18.3 ^{a} | NA | NA |
| Residence Time | (jump dilution vs. 50 μM ATP) | | | |
| | BTK Kᵢ, nM | 7.1 ^{d} | NA | NA |
| | BTK Kᵢ*, nM | 0.17 ^{d} | NA | NA |
| | BTK residence time, h | 6.6 ^{d} | NA | NA |
| | (competitive binding) | | | |
| Whole Blood | CD69 Whole Human Blood IC₅₀, nM | 8.4 ^{a} | 84 ^{b} | 10 ^{c} |
| | CD63 Whole Human Blood IC₅₀, nM | 30.7 ^{a} | 1,660 ^{b} | 166 ^{c} |

^{a}Crawford, et al., J Med Chem 2018, 61: 2227-2245
^{b}Haselmeyer, J Immunol 2019, 202: 2888-2906
^{c}Francesco, ECTRIMS 2017 poster (PRN), available at <https://onlinelibrary.ectrims-congress.eu/ectrims/2017/ACTRIMS-ECTRIMS2017/200644/michelle.r.francesco.pm2246.a.potent.and.selective.blood.brain.barrier.html>
^{d}unpublished
NA = not applicable

Unpublished kinase selectivity data were obtained generally following the procedures of Crawford, et al., J Med Chem 2018, 61: 2227-2245 (SI pp S31-S32). Unpublished covalent reaction (k_{inact}/Kᵢ) data were obtained generally following the procedures of Schnute, et al., ACS Med Chem Lett 2018, 10: 80-85 (SI pp S29-S31), using N-terminal His-tagged full-length recombinant human BTK. BTK residence time data were obtained following the procedures of Crawford, et al., J Med Chem 2018, 61: 2227-2245 (SI pp S43). Unpublished competitive binding kinetics data were obtained generally following the procedures of Schnute, et al., ACS Med Chem Lett 2018, 10: 80-85 (SI pp S29-S31), using N-terminal His-tagged full-length recombinant human BTK. The impact of ibrutinib, another covalent BTK inhibitor, on activation of B cells and basophils in human whole blood was also assessed (CD63 IC₅₀ nM=171; CD69 IC₅₀ nM=12; Crawford, et al., J Med Chem 2018, 61: 2227-2245).

What is claimed is:

1. A method of treating primary progressive multiple sclerosis (PPMS) in a subject in need thereof, comprising administering to the subject about 200 mg fenebrutinib twice daily, or an equivalent amount of a pharmaceutically acceptable salt thereof.

2. The method of claim 1, further comprising evaluating disability progression in the subject, wherein disability progression is evaluated using the Expanded Disability Status Scale (EDSS), the 9-Hole Peg Test (9-HPT), or the Timed 25-Foot Walk Test (T25FWT), or any combinations thereof.

3. The method of claim 1 or 2, further comprising evaluating the onset of composite 12-week confirmed disability progression (cCDP12), wherein onset of the cCDP12 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.
and wherein the progression event is confirmed at least 12 weeks after the initial progression.

4. The method of any one of claims 1 to 3, wherein time to a progression event in the subject is increased, wherein the progression event is:
an increase from baseline in EDSS score of at least 1.0 point in a subject with a baseline EDSS score of less than or equal to 5.5 points; or
an increase from baseline in EDSS score of at least 0.5 point in a subject with a baseline EDSS score of greater than 5.5 points.

5. The method of any one of claims 1 to 4, wherein time to a progression event in the subject is increased, wherein the progression event is increase of at least 20% from baseline in time to complete the 9-HPT.

6. The method of any one of claims 1 to 5, wherein time to a progression event in the subject is increased, wherein the progression event is an increase of at least 20% from baseline in T25FWT.

7. The method of any one of claims 1 to 6, wherein time to onset of CDP12, cCDP12, CDP24, or cCDP24 is increased in comparison to a subject with PPMS who is not administered fenebrutinib or a pharmaceutically acceptable salt thereof.

8. The method of claim 7, wherein the subject with PPMS who is not administered fenebrutinib is administered an anti-CD20 antibody.

9. The method of any one of claims 4 to 8, wherein the time to a progression event or time to onset is increased at least 10%.

10. A method of slowing the progression of PPMS in a subject in need thereof, comprising administering to the subject about 200 mg fenebrutinib twice per day, or an equivalent amount of a pharmaceutically acceptable salt thereof.

11. The method of claim 10, wherein the progression of PPMS is evaluated using the MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels.

12. The method of claim 10 or 11, wherein the progression of PPMS comprises at least one progression event.

13. A method of delaying the onset of at least one progression event in a subject with PPMS, the method comprising administering to the subject about 200 mg fenebrutinib twice daily, or an equivalent amount of a pharmaceutically acceptable salt thereof.

14. A method of reducing the risk of a subject with PPMS having at least one progression event, the method comprising administering to the subject about 200 mg fenebrutinib twice daily, or an equivalent amount of a pharmaceutically acceptable salt thereof.

15. The method of any one of claims 12 to 14, wherein the at least one progression event is selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.

16. The method of claim 15, wherein the progression event is confirmed at least 12 weeks after the initial progression.

17. The method of any one of claims 1 to 12, 15, or 16, wherein the progression of PPMS in the subject is slowed by at least 10% compared to another subject with PPMS who is not administered fenebrutinib or a pharmaceutically acceptable salt thereof and is administered an anti-CD20 antibody.

18. The method of any one of claims 13, 15, or 16, wherein the onset of at least one progression event is delayed by at least 10% compared to another subject with PPMS who is not administered fenebrutinib or a pharmaceutically acceptable salt thereof and is administered an anti-CD20 antibody.

19. The method of any one of claims 1 to 16, wherein the risk of the subject having at least one progression event is decreased by at least 10% compared to another subject with PPMS who is not administered fenebrutinib or a pharmaceutically acceptable salt thereof and is administered an anti-CD20 antibody.

20. A method of reducing disability in a subject with PPMS, the method comprising administering to the subject about 200 mg fenebrutinib twice per day, or an equivalent amount of a pharmaceutically acceptable salt thereof.

21. The method of claim 20, wherein reducing disability comprises:
reducing the psychological impact of MS;
increasing upper limb function;
increasing walking ability;
decreasing fatigue;
improving work status; or
decreasing global impression of MS severity;
or any combinations thereof.

22. The method of any one of claims 1 to 21, wherein the subject has a reduction in one or more symptoms of PPMS after beginning treatment with fenebrutinib, or a pharmaceutically acceptable salt thereof.

23. The method of any one of claims 1 to 22, wherein the method further comprises the step of measuring one or more clinical or laboratory endpoints in the subject in order to evaluate the efficacy of treating PPMS.

24. The method of claim 23, wherein the one or more clinical or laboratory endpoints are selected from the group consisting of the subject's MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels.

25. The method of any one of claims 1 to 24, wherein the fenebrutinib or pharmaceutically acceptable salt thereof is administered orally.

26. The method of any one of claims 1 to 25, wherein the fenebrutinib or pharmaceutically acceptable salt thereof is administered in the form of one or more tablets or capsules.

27. The method of any one of claims 1 to 26, wherein the fenebrutinib or pharmaceutically acceptable salt thereof is administered in the form of two tablets twice daily, each tablet comprising about 100 mg fenebrutinib or an equivalent amount of a pharmaceutically acceptable salt thereof.

28. The method of any one of claims 1 to 27, wherein the free form of fenebrutinib is administered.

29. A compound for use in a method of treating primary progressive multiple sclerosis (PPMS) in a subject in need thereof, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

30. The compound for use of claim 29, wherein the method further comprises evaluating disability progression in the subject, wherein disability progression is evaluated using the Expanded Disability Status Scale (EDSS), the 9-Hole Peg Test (9-HPT), or the Timed 25-Foot Walk Test (T25FWT), or any combinations thereof.

31. The compound for use of claim 29 or 30, wherein the method further comprises evaluating the onset of composite 12-week confirmed disability progression (cCDP12), wherein onset of the cCDP12 comprises at least one progression event selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.
and wherein the progression event is confirmed at least 12 weeks after the initial progression.

32. The compound for use of any one of claims 29 to 31, wherein time to a progression event is increased, wherein the progression event is:
an increase from baseline in EDSS score of at least 1.0 point in a subject with a baseline EDSS score of less than or equal to 5.5 points; or
an increase from baseline in EDSS score of at least 0.5 point in a subject with a baseline EDSS score of greater than 5.5 points.

33. The compound for use of any one of claims 29 to 32, wherein time to a progression event is increased, wherein the progression event is increase of at least 20% from baseline in time to complete the 9-HPT.

34. The compound for use of any one of claims 29 to 33, wherein time to a progression event is increased, wherein the progression event is an increase of at least 20% from baseline in T25FWT.

35. The compound for use of any one of claims 29 to 34, wherein time to onset of CDP12, cCDP12, CDP24, or cCDP24 is increased in comparison to a subject with PPMS who is not administered fenebrutinib or a pharmaceutically acceptable salt thereof.

36. The compound for use of claim 35, wherein the subject with PPMS who is not administered fenebrutinib is administered an anti-CD20 antibody.

37. The compound for use of any one of claims 32 to 36, wherein the time to a progression event or time to onset is increased at least 10%.

38. A compound for use in a method of slowing the progression of PPMS in a subject in need thereof, wherein the compound is fenebrutinib or a pharmaceutically acceptable salt thereof, and wherein the compound comprises administering to the subject about 200 mg fenebrutinib twice per day, or an equivalent amount of a pharmaceutically acceptable salt thereof.

39. The compound for use of claim 38, wherein the progression of PPMS is evaluated using the MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels.

40. The compound for use of claim 38 or 39, wherein the progression of PPMS comprises at least one progression event.

41. A compound for use in a method of delaying the onset of at least one progression event in a subject with PPMS, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

42. A compound for use in a method of reducing the risk of a subject with PPMS having at least one progression event, wherein the compound is fenebrutinib or a pharmaceutically acceptable salt thereof, and wherein the method comprises administering to the subject about 200 mg fenebrutinib twice daily, or an equivalent amount of a pharmaceutically acceptable salt thereof.

43. The compound for use of any one of claims 40 to 42, wherein the at least one progression event is selected from the group consisting of:
(a) an increase from baseline in EDSS score of at least 1.0 point in a subject with a baseline EDSS score of less than or equal to 5.5 points; or an increase from baseline in EDSS score of at least 0.5 point in a subject with a baseline EDSS score of greater than 5.5 points;
(b) increase from baseline of at least 20% in time to complete the 9-HPT; and
(c) increase from baseline of at least 20% in T25FWT.

44. The compound for use of claim 43, wherein the progression event is confirmed at least 12 weeks after the initial progression.

45. The compound for use of any one of claims 38 to 40, 43, or 44, wherein the progression is slowed by at least 10% compared to another subject with PPMS who is not administered fenebrutinib or a pharmaceutically acceptable salt thereof and is administered an anti-CD20 antibody.

46. The compound for use of any one of claims 41, 43, or 44, wherein the onset of at least one progression event is delayed by at least 10% compared to another subject with PPMS who is not administered fenebrutinib or a pharmaceutically acceptable salt thereof and is administered an anti-CD20 antibody.

47. The compound for use of any one of claims 42 to 44, wherein the risk of having at least one progression event is decreased by at least 10% compared to another subject with PPMS who is not administered fenebrutinib or a pharmaceutically acceptable salt thereof and is administered an anti-CD20 antibody.

48. The compound for use of any one of claims 38 to 47, wherein the progression is slowed, or the onset is delayed, or the risk is decreased, in comparison to a subject with PPMS that is not administered fenebrutinib or a pharmaceutically acceptable salt thereof.

49. A compound for use in a method of reducing disability in a subject with PPMS, wherein the compound is fenebrutinib or a pharmaceutically acceptable salt thereof, and wherein the method comprises administering to the subject about 200 mg fenebrutinib twice per day, or an equivalent amount of a pharmaceutically acceptable salt thereof.

50. The compound for use of claim 49, wherein reducing disability comprises:
reducing the psychological impact of MS;
increasing upper limb function;
increasing walking ability;
decreasing fatigue;
improving work status; or
decreasing global impression of MS severity;
or any combinations thereof.

51. The compound for use of any one of claims 29 to 50, wherein the subject has a reduction in one or more symptoms of PPMS after beginning treatment with fenebrutinib, or a pharmaceutically acceptable salt thereof.

52. The compound for use of any one of claims 29 to 51, wherein the method further comprises the step of measuring one or more clinical or laboratory endpoints in the subject in order to evaluate the efficacy of treating PPMS.

53. The compound for use of claim 52, wherein the one or more clinical or laboratory endpoints are selected from the group consisting of the subject's MSIS-29, Neuro-QoL Upper Extremity, PROMIS-Fatigue_{MS}, MSWS-12, PGI-S, WPAI:MS, PGI-C, EQ-5D-5L, C-SSRS, 9-HPT, T25FWT, EDSS, SDMT, MRI, or NfL levels.

54. The compound for use of any one of claims 29 to 53, wherein the fenebrutinib or pharmaceutically acceptable salt thereof is administered orally.

55. The compound for use of any one of claims 29 to 54, wherein the fenebrutinib or pharmaceutically acceptable salt thereof is administered in the form of one or more tablets or capsules.

56. The compound for use of any one of claims 29 to 55, wherein the fenebrutinib or pharmaceutically acceptable salt thereof is administered in the form of two tablets twice daily, each tablet comprising about 100 mg fenebrutinib or an equivalent amount of a pharmaceutically acceptable salt thereof.

57. The compound for use of any one of claims 29 to 56, wherein the free form of fenebrutinib is administered.

58. The method of any one of claims 1 to 28, or the compound for use of any one of claims 29 to 57, wherein the subject with PPMS has had progressive disease from the onset, and has been in a progressive stage for at least 12 months prior to beginning administration of fenebrutinib or a pharmaceutically acceptable salt thereof.

59. The method of any one of claims 1 to 28 or 58, or the compound for use of any one of claims 29 to 58, wherein prior to beginning administration of fenebrutinib or a pharmaceutically acceptable salt thereof, the subject has at least two of:
(a) one or more T2-hyperintense lesions characteristic of MS in one or more of the periventricular, cortical or juxtacortical, or infratentorial the following brain regions;
(b) two or more T2-hyperintense lesions in the spinal cord; and
(c) the presence of cerebrospinal fluid-specific oligoclonal bands.

60. The method of any one of claims 1 to 28, 58, or 59, or the compound for use of any one of claims 29 to 59, wherein the subject has an EDSS score from 3.0 to 6.5 prior to beginning administration of fenebrutinib or a pharmaceutically acceptable salt thereof.

61. The method of any one of claims 1 to 28 or 58 to 60, or the compound for use of any one of claims 29 to 60, wherein the subject with PPMS does not have one or more of:
estimated glomerular filtration rate (eGFR)<60 mL/min/1.73 m²;
ALT or AST>2×ULN;
total bilirubin greater than 1.5×ULN;
hemoglobin<9.5 g/dL;
platelet count<100×10⁹/L; or
abnormalities in one or more of the hepatic synthetic function tests PT, INR, PTT, or albumin.

62. The method of any one of claims 1 to 28 or 58 to 61, or the compound for use of any one of claims 29 to 61, wherein the subject is not concomitantly administered a strong CYP3A4 inhibitor while being administered about 200 mg fenebrutinib twice per day, or an equivalent amount of a pharmaceutically acceptable salt thereof.

63. The method of any one of claims 1 to 28 or 58 to 62, or the compound for use of any one of claims 29 to 62, wherein the subject is not concomitantly administered a strong CYP3A4 inducer while being administered about 200 mg fenebrutinib twice per day, or an equivalent amount of a pharmaceutically acceptable salt thereof.

64. The method of any one of claims 1 to 28 or 58 to 63, or the compound for use of any one of claims 29 to 63, wherein the subject is not concomitantly administered a moderate CYP3A4 inducer while being administered about 200 mg fenebrutinib twice per day, or an equivalent amount of a pharmaceutically acceptable salt thereof.

65. The method of any one of claims 1 to 28 or 58 to 64, or the compound for use of any one of claims 29 to 64, wherein the subject is not concomitantly administered a CYP3A4 substrate with a narrow therapeutic window while being administered about 200 mg fenebrutinib twice per day, or an equivalent amount of a pharmaceutically acceptable salt thereof.

66. A compound for use in the manufacture of a medicament for any of the methods of claims 1 to 28 or 58 to 65, wherein the compound is fenebrutinib or a pharmaceutically acceptable salt thereof.
